(19) United States
(12) Patent Application Publication
(10) Pub. No.: US 2017/0044552 A1
(43) Pub. Date: Feb. 16, 2017

(54) Methods for Recombinant Production of Saffron Compounds

(71) Applicant: EVOLVA SA, Reinach (CH)

(72) Inventor: A.S. Sathish Kumar, Neelankarai Chennai (IN)

(21) Appl. No.: 15/305,582

(22) PCT Filed: Apr. 24, 2015
(86) PCT No.: PCT/EP2015/058994

§ 371 Date: Oct. 20, 2016

Related U.S. Application Data
(60) Provisional application No. 61/984,582, filed on Apr. 25, 2014.

(51) Int. Cl. C12N 15/52; C12N 9/02; C12P 19/46; C12P 7/24

(52) U.S. Cl. CPC C12N 15/52; C12P 7/24; C12N 9/0071; C12N 9/0095; C12Y 118/01002; C12P 19/46

(57) ABSTRACT

Recombinant microorganisms and methods for producing saffron compounds including hydroxy-β-cylcocitral and picrocrocin are disclosed herein. Methods involve expression of a gene encoding a cytochrome p450 polypeptide, and optionally a gene encoding a (2Fe-2S) ferredoxin polypeptide, a gene encoding a flavin-dependent ferredoxin reductase, and a gene encoding a uridine 5′-diphospho glycosyltransferase (UGT) polypeptide.

### BACKGROUND OF THE INVENTION

Field of the Invention

The invention disclosed herein relates generally to the field of genetic engineering. Particularly, the invention disclosed herein provides methods and materials for recombinantly producing flavorant, aromatic, and colorant compounds from Crocus sativus, the saffron plant.

Description of Related Art

Saffron is a dried spice obtained by extraction from the stigma of the Crocus sativus L. flower and is considered to have been employed for human use for over 3500 years. Saffron has historically been used medicinally, but in recent times, it is largely utilized for its colorant properties. The main pigment of saffron is crocin, which is a mixture of glycosides that impart yellowish red colors. A major constituent of crocin is α-crocin, which is yellow in color. Crocetin, another one of the major components of saffron, has antioxidant properties similar to related carotenoid-type molecules and is a colorant. Other glycosidic forms of crocetin (also called α-crocetin or crocetin-l) include α-crocetin gentiobioside, glucoside, gentioglucoside, and diglucoside. Y-crocetin in the mono- or di-methylester form that is also present in saffron, along with β-cis-crocetin and trans-crocetin isomers. Safranal (4-hydroxy-2,4,4-trimethyl 1-cyclohexene-1-carboxaldehyde, or dehydro-β-cyclocitral) is thought to be a product of the drying process and has odorant qualities as well that can be utilized in food preparation. Picrocrocin, which is colorless, is the bitter part of the saffron extracts. Thus, saffron extracts are used for many purposes, as a colorant or a flavorant, or for its odorant properties.

The saffron plant is grown commercially in many countries including Italy, France, India, Spain, Greece, Morocco, Turkey, Switzerland, Israel, Pakistan, Azerbaijan, China, Egypt, United Arab Emirates, Japan, Australia, and Iran. Iran produces approximately 80% of the total world annual saffron production (estimated to be just over 200 tons). It has been reported that over 150,000 flowers are required for 1 kg of product. Plant breeding efforts to increase yields are complicated by the triploidy of the plant's genome, resulting in sterile plants. In addition, the plant is in bloom only for about 15 days starting in middle or late October. Typically, production involves manual removal of the stigmas from the flower which is also an inefficient process. Selling prices of over $1000/kg of saffron are typical. Therefore, there remains a need for an alternative bio-conversion or de novo biosynthesis of the components of saffron.

### SUMMARY OF THE INVENTION

It is against the above background that the present invention provides certain advantages and advancements over the prior art.

The invention disclosed herein is based on the discovery of methods and materials for improving production of useful compounds from Crocus sativus, the saffron plant, in recombinant hosts, as well as nucleotides and polypeptides useful in establishing recombinant pathways for production of compounds such as hydroxyl-β-cyclocitral or such as glycosylated saffron compound, picrocrocin. These products can be produced singly and recombined for optimal characteristics in a food system or for medicinal supplements or, optionally, be produced as a mixture. In some aspects, the host is recombinant yeast.

Any of the hosts described herein can include an exogenous nucleic acid encoding one or more of a cytochrome p450, a (2Fe-2S) ferredoxin, a flavin-dependent ferredoxin reductase, and a uridine 5-′diphospho glcosyltransferase.

In some aspects, the cytochrome p450 is Novosphingobium aromaticivorans CYP101B1.

In some aspects, the (2Fe-2S) ferredoxin is truncated Novosphingobium aromaticivorans ArX, tArX.

In some aspects, the flavin-dependent ferredoxin reductase is Novosphingobium aromaticivorans ArR.

In some aspects, the uridine 5′-diphospho glycosyltransferase gene is Stevia rebaudiana 73EV12.

Any of the hosts described herein can produce hydroxyl-β-cyclocitral and/or picrocrocin.

In some aspects, recombinant DNA constructs disclosed herein comprise DNA molecules disclosed herein, wherein the DNA molecules are operably linked to a promoter, wherein the promoter is a promoter from a gene including but not limited to glyceraldehyde-3-phosphate dehydrogenase (GPD), triose phosphate isomerase (TPI), galactose (GAL), phosphoglycerate kinase (PGK), cytochrome c (CYC), kexin (KEX), translation elongation factor (TEF), pyruvate decarboxylase (PDC), pyruvate kinase (PYK), thermostable direct hemolysin (TDH), fructose-bisphosphate aldolase (FBA), hexose transporter (HXT7), alcohol dehydrogenase (ADH) and variants thereof (see, for example, SEQ IDs 9-15; FIG. 7).

In some aspects, expression vectors comprise the recombinant DNA constructs disclosed herein.

Any of the hosts described herein can be a microorganism, a plant, or a plant cell. The microorganism can be a Saccharomycete such as Saccharomyces cerevisiae or Escherichia coli. The plant or plant cell can be Crocus sativus. A recombinant host disclosed herein can be a yeast cell, a plant cell, a mammalian cell, an insect cell, a fungal cell, an algal cell, a cyanobacteria or a bacterial cell.

In some aspects, the yeast cell is a cell from Saccharomyces cerevisiae, Schizosaccharomyces pombe, Yarrowia lipolytica, Candida glabrata, Ashbya gossypfi, Cyberlindnera jadinfi, Pichia pastoris, Kluyveromyces lactis, Hansenula polymorpha, Candida boidinfi, Arxula adeninivorans, Xanthophyllomyces dendrorhous, Candida albicans, Rhodotorula sp., or Rhodospiridium sp.

In some aspects, the yeast cell is a Saccharomycete.

In some aspects, the yeast cell is a Saccharomyces cerevisiae cell.

In some aspects, the algal cell is a cell from Blakeslea trispora, Dunaliella sauna, Haematococcus pluvialis, Chlorella sp., Undaria pinnatifida, Sargassum, Laminaria japonica, Scenedesmus almeriensis species.

In some aspects, the cyanobacerial cell is a cell from Phormidium laminosum, Microcystis sp., Synechococcus sp., Pantoea sp., Flavobacterium sp.

In some aspects, the recombinant host disclosed herein is cultured in the presence of β-cyclocitral.

Although this invention as disclosed herein is not limited to specific advantages or functionalities, the invention generally provides recombinant host cells that express a gene encoding a cytochrome p450 polypeptide; a gene encoding a (2Fe-2S) ferredoxin polypeptide; a gene encoding a flavin-dependent ferredoxin reductase, wherein at least one of said genes is a recombinant gene.

In some aspects, the gene encoding a cytochrome p450 polypeptide is Novosphingobium aromaticivorans CYP101B1, set forth in SEQ ID NO:1.

In some aspects, the cytochrome p450 polypeptide comprises a cytochrome p450 polypeptide having 40% or greater identity to the amino acid sequence set forth in SEQ ID NO:2.

In some aspects, the (2Fe-2S) ferredoxin gene is truncated Novosphingobium aromaticivorans Arx (tARX) set forth in SEQ ID NO:3.

In some aspects, the (2Fe-2S) ferredoxin polypeptide comprises a (2Fe-2S) ferredoxin polypeptide having 40% or greater identity to the amino acid sequence set forth in SEQ ID NO:4.

In some aspects, the flavin-dependent ferredoxin reductase gene is Novosphingobium aromaticivorans ArR set forth in SEQ ID NO:5.

In some aspects, the flavin-dependent ferredoxin reductase polypeptide comprises a flavin-dependent ferredoxin reductase polypeptide having 40% or greater identity to the amino acid sequence set forth in SEQ ID NO:6.

In some aspects, the recombinant host disclosed herein produces hydroxy-8-cyclocitral, wherein the recombinant host expresses one or a plurality of genes wherein the plurality of genes are CYP101B1, ArX, tArR, CrtI, CrtE, CrtYB, CCD6, ALD9 and UGT75L6.

In some aspects, the recombinant host disclosed herein further comprises a recombinant gene encoding a uridine 5′-diphospho glycosyltransferase (UGT) polypeptide.

In some aspects, the gene is Stevia rebaudiana 73EV12 set forth in SEQ ID NO:7.

In some aspects, the UGT polypeptide comprises a polypeptide having 40% or greater identity to the amino acid sequence set forth in SEQ ID NO:8.

In some aspects, the recombinant host disclosed herein produces picrocrocin wherein the recombinant host expresses one or a plurality of genes wherein the plurality of genes are CYP101B1, 73EV12, ArX, tArR, CrtI, CrtE, CrtYB, CCD6, ALD9 and UGT75L6.

The invention further provides a recombinant DNA molecule encoding a cytochrome p450 polypeptide; a (2Fe-2S) ferredoxin polypeptide; and a flavin-dependent ferredoxin reductase.

In some aspects, the cytochrome p450 polypeptide comprises a cytochrome p450 polypeptide having 40% or greater identity to the amino acid sequence set forth in SEQ ID NO:2; the (2Fe-2S) ferredoxin polypeptide comprises a (2Fe-2S) ferredoxin polypeptide having 40% or greater identity to the amino acid sequence set forth in SEQ ID NO:4; and the flavin-dependent ferredoxin reductase polypeptide comprises a flavin-dependent ferredoxin reductase polypeptide having 40% or greater identity to the amino acid sequence set forth in SEQ ID NO:6.

The invention further provides a recombinant host comprising one or more expression vectors as disclosed herein, wherein the cell produces hydroxyl-β-cyclocitral.

In some aspects, the cell comprises a gene encoding a cytochrome p450 polypeptide; a gene encoding a (2Fe-2S) ferredoxin polypeptide; and a gene encoding a flavin-dependent ferredoxin reductase.

In some aspects, the gene encoding a cytochrome p450 polypeptide is Novosphingobium aromaticivorans CYP101B1, set forth in SEQ ID NO:1; the [2Fe-2S] ferredoxin gene is truncated Novosphingobium aromaticivorans Arx (tARX) set forth in SEQ ID NO:3, and the flavin-dependent ferredoxin reductase gene is Novosphingobium aromaticivorans ArR set forth in SEQ ID NO:5.

The invention further provides a recombinant DNA molecule encoding a cytochrome p450 polypeptide; a (2Fe-2S) ferredoxin polypeptide; a flavin-dependent ferredoxin reductase; and a UGT polypeptide.

In some aspects, the cytochrome p450 polypeptide comprises a cytochrome p450 polypeptide having 40% or greater identity to the amino acid sequence set forth in SEQ ID NO:2; the (2Fe-2S) ferredoxin polypeptide comprises a (2Fe-2S) ferredoxin polypeptide having 40% or greater identity to the amino acid sequence set forth in SEQ ID NO:4; the flavin-dependent ferredoxin reductase polypeptide comprises a flavin-dependent ferredoxin reductase polypeptide having 40% or greater identity to the amino acid sequence set forth in SEQ ID NO:6; and the UGT polypeptide comprises a polypeptide having 40% or greater identity to the amino acid sequence set forth in SEQ ID NO:8.

The invention further provides a recombinant host comprising one or more expression vectors as disclosed herein, wherein the cell produces picrocrocin.

In some aspects, the recombinant host comprises a gene encoding a cytochrome p450 polypeptide; a gene encoding a (2Fe-2S) ferredoxin polypeptide; a gene encoding a flavin-dependent ferredoxin reductase; and a gene encoding UGT polypeptide.

In some aspects, the gene encoding a cytochrome p450 polypeptide is Novosphingobium aromaticivorans CYP101B1, set forth in SEQ ID NO:1; the (2Fe-2S) ferredoxin gene is truncated Novosphingobium aromaticivorans Arx (tARX), set forth in SEQ ID NO:3, the flavin-dependent ferredoxin reductase gene is Novosphingobium aromaticivorans ArR set forth in SEQ ID NO:5; and the gene encoding UGT polypeptide is Stevia rebaudiana 73EV12 set forth in SEQ ID NO:7.

The invention further provides a method of producing a saffron compound, comprising cultivating the recombinant host cell as disclosed herein in a culture medium under conditions in which a gene encoding a cytochrome p450 polypeptide; a gene encoding a (2Fe-2S) ferredoxin polypeptide; and a gene encoding a flavin-dependent ferredoxin reductase are expressed, wherein the saffron compound is hydroxyl-β-cyclocitral.

In some aspects of the methods disclosed herein, the gene encoding a cytochrome p450 polypeptide has an amino acid sequence set forth in SEQ ID NO:2; the (2Fe-2S) ferredoxin gene has an amino acid sequence set forth in SEQ ID NO:4, and the flavin-dependent ferredoxin reductase gene has an amino acid sequence set forth in SEQ ID NO:6.

The invention further provides a method of producing a saffron compound, comprising cultivating a recombinant host as disclosed herein in a culture medium under conditions in which a gene encoding a cytochrome p450 polypeptide; a gene encoding a (2Fe-2S) ferredoxin polypeptide; a gene encoding a flavin-dependent ferredoxin reductase; and a gene encoding UGT polypeptide are expressed; wherein the saffron compound is a glycosylated saffron compound, wherein the glycosylated saffron compound is picrocrocin.

In some aspects of the methods disclosed herein, the gene encoding a cytochrome p450 polypeptide has an amino acid sequence set forth in SEQ ID NO:2; the (2Fe-2S) ferredoxin gene has an amino acid sequence set forth in SEQ ID NO:4, the flavin-dependent ferredoxin reductase gene has an amino acid sequence set forth in SEQ ID NO: 6; and the gene encoding UGT polypeptide has an amino acid sequence set forth in SEQ ID NO:8.

The invention further provides a method of producing a saffron compound, comprising cultivating the recombinant host as disclosed herein in a culture medium under conditions in which the gene encoding a cytochrome p450 polypeptide is expressed, wherein the saffron compound is hydroxyl-β-cyclocitral.

The invention further provides a method of producing a saffron compound, comprising cultivating the recombinant host as disclosed herein in a culture medium under conditions in which the gene encoding a cytochrome p450 polypeptide and the gene encoding UGT polypeptide are expressed, wherein the saffron compound is a glycosylated saffron compound, wherein the glycosylated saffron compound is picrocrocin.

In some aspects of the methods disclosed herein, wherein the recombinant host comprises a yeast cell, a plant cell, a mammalian cell, an insect cell, a fungal cell, algal cell, cyanobacteria or a bacterial cell.

In some aspects of the methods disclosed herein, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

In some aspects of the methods disclosed herein, the yeast cell is a Saccharomycete.

In some aspects of the methods disclosed herein, the yeast cell is a Saccharomyces cerevisiae cell.

In some aspects of the methods disclosed herein, the algal cell is a cell from Blakeslea trispora, Dunaliella salina, Haematococcus pluvialis, Chlorella sp., Undaria pinnatifida, Sargassum, Laminaria japonica, Scenedesmus almeriensis species.

In some aspects of the methods disclosed herein, the cyanobacerial cell is a cell from Phormidium laminosum, Microcystis sp., Synechococcus sp., Pantoea sp., Flavobacterium sp.

The invention disclosed herein further provides methods for producing a saffron compound, comprising growing a recombinant host as disclosed herein in a culture medium under conditions in which one or a plurality of genes is expressed, wherein said saffron-related compound is hydroxyl-β-cyclocitral or picrocrocin and wherein the plurality of genes are CYP101B1, ArX, tArR, 73EV12, CrtI, CrtE, CrtYB, CCD6, ALD9 and UGT75L6.

In particular aspects, a recombinant host used in methods disclosed herein is cultivated using a fermentation process.

The invention disclosed herein further provides a recombinant host that expresses a gene encoding a cytochrome p450 polypeptide wherein the gene encoding the cytochrome p450 polypeptide is Streptomyces avermitilis CYP102D1, set forth in SEQ ID NO: 22.

In some aspects, the cytochrome p450 polypeptide comprises a cytochrome p450 polypeptide having 40% or greater identity to the amino acid sequence set forth in SEQ ID NO: 23.

In some aspects, the cell disclosed herein further comprises a gene encoding a uridine 5′-diphospho glycosyltransferase (UGT) polypeptide.

In some aspects, the uridine 5′-diphospho glycosyltransferase gene is Stevia rebaudiana 73EV12 set forth in SEQ ID NO:7.

In some aspects, the UGT polypeptide comprises a polypeptide having 40% or greater identity to the amino acid sequence set forth in SEQ ID NO:8.

As such, any of the hosts described herein can produce hydroxyl-β-cyclocitral or picrocrocin.

Any of the hosts described herein can be a microorganism, a plant, or a plant cell. The microorganism can be a Saccharomycete such as Saccharomyces cerevisiae or Escherichia coli. The plant or plant cell can be Crocus sativus.

The invention disclosed herein further provides a method of producing a saffron compound, comprising growing the recombinant host as disclosed herein in a culture medium under conditions in which one or a plurality of genes is expressed, wherein said saffron-related compound is selected from the group comprising hydroxyl-β-cyclocitral and picrocrocin and wherein the plurality of genes are CYP102D1, 73EV12, CrtI, CrtE, CrtYB, CCD6, ALD9 and UGT75L6.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of this invention can be best understood when read in conjunction with the following drawings, where like structure is indicated with like reference numerals and in which:

FIG. 1A shows production of β-carotene from glucose and intermediates. FIG. 1B is a schematic of the biosynthetic pathway from β-carotene to picrocrocin. FIG. 10 is a schematic of additional biosynthetic pathways from β-carotene to saffron compounds such as picrocrocin, crocetin, and crocetin dialdehyde.

FIG. 2 shows a high-performance liquid chromatography (HPLC) spectrum of hydroxyl-β-cyclocitral produced from β-cyclocitral.

FIG. 3A shows a gas chromatography (GC) spectrum of hydroxyl-β-cyclocitral produced from β-cyclocitral. FIG. 3B shows a mass spectroscopy (MS) spectrum of hydroxyl-β-cyclocitral produced from β-cyclocitral.

FIG. 4A shows a liquid chromatography (LC) spectrum of picrocrocin produced from β-cyclocitral. FIG. 4B shows an MS spectrum of picrocrocin produced from β-cyclocitral.

FIG. 5A shows the pETDuet-1 vector. FIG. 5B shows the RSFDuet-1 vector.

FIG. 6 contains the nucleotide and amino acid sequences of CYP101B1 (SEQ ID NO: 1 and SEQ ID NO: 2), tArX (SEQ ID NO: 3 and SEQ ID NO: 4), ArR (SEQ ID NO: 5 and SEQ ID NO: 6), 73EV12 (SEQ ID NO: 7 and SEQ ID NO: 8), CCD5 (SEQ ID NO: 16 and NO: 17), CCD6 (SEQ ID NO: 18 and NO:19), and CCD1a (SEQ ID NO: 20 and NO: 21).

FIG. 7 shows the sequences of yeast constitutive promoters GPD (TDH3), CYC, ADH1, mid-length ADH1, PGK1, Ste5, and CLB1.

FIG. 8 shows pEVE2262 (pESC-LEU PGK1 TFF1) vector.

FIG. 9 shows a high-performance liquid chromatography (HPLC) spectrum of hydroxy-8-cyclocitral and picrocrocin produced using the 2C yeast strain.

FIG. 10 contains the nucleotide and amino acid sequences of CYP102D1 (SEQ ID NO: 22 and SEQ ID NO: 23).

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the terms “substantial” or “substantially” are utilized herein to represent the inherent degree of uncertainty that can be attributed to any quantitative comparison, value, measurement, or other representation. The terms “substantial” or “substantially” are also utilized herein to represent the degree by which a quantitative representation can vary from a stated reference without resulting in a change in the basic function of the subject matter at issue.

As used herein, saffron compounds can include, but are not limited to, crocin, which is a mixture of glycosides that impart yellowish red colors and in particular α-crocin, which is yellow in color; crocetin (also called α-crocetin or crocetin-l) and glycosidic forms of crocetin that include α-crocetin gentiobioside, glucoside, gentioglucoside, and diglucoside; Y-crocetin in the mono- or di-methylester form; β-cis-crocetin and trans-crocetin isomers; safranal (4-hydroxy-2,4,4-trimethyl 1-cyclohexene-1-carboxaldehyde, or dehydro-β-cyclocitral), hydroxyl-β-cyclocitral and picrocrocin.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

In particular aspects, recombinant hosts such as microorganisms are provided that can express genes coding for polypeptides useful in the biosynthesis of saffron compounds. Expression of these biosynthetic polypeptides in various microbial chassis allows saffron compounds to be produced in a consistent, reproducible manner from energy and carbon sources such as sugars, glycerol, CO₂, H₂, and sunlight. The proportion of each compound produced by a recombinant host can be tailored by incorporating preselected biosynthetic enzymes into the hosts and expressing them at appropriate levels.

At least one of the genes can be a recombinant gene, the particular recombinant gene(s) depending on the species or strain selected for use. Additional genes or biosynthetic modules can be included in order to increase compound yield, improve efficiency with which energy and carbon sources are converted to saffron compounds, and/or to enhance productivity from the cell culture or plant. Such additional biosynthetic modules include genes involved in the synthesis of the terpenoid precursors, isopentenyl diphosphate and dimethylallyl diphosphate.

In certain aspects of this invention, the recombinant host comprises a yeast cell, a plant cell, a mammalian cell, an insect cell, a fungal cell, an algal cell, a cyanobacteria or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

In some aspects, the yeast cell is a Saccharomycete.

In some aspects, the yeast cell is a Saccharomyces cerevisiae cell.

In some aspects, the algal cell is a cell from Blakeslea trispora, Dunaliella salina, Haematococcus pluvialis, Chlorella sp., Undaria pinnatifida, Sargassum, Laminaria japonica, Scenedesmus almeriensis species.

In some aspects, the cyanobacerial cell is a cell from Phormidium laminosum, Microcystis sp., Synechococcus sp., Pantoea sp., Flavobacterium sp.

In certain aspects of this invention, microorganisms can include, but are not limited to, S. cerevisiae and E. coli. The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

In particular aspects, a recombinant host comprises a cytochrome p450 class I electron transfer system. In particular aspects, the cytochrome p450 class I electron transfer system comprises genes encoding cytochrome p450, ferredoxin, and flavin-dependent ferredoxin reductase polypeptides.

In some aspects, a cytochrome p450 converts β-cyclocitral to hydroxyl-β-cyclocitral. Non-limiting examples of such cytochrome p450 enzymes are shown in Table 1.

TABLE 1
Examples of cytochrome p450 enzymes.

| Cytochrome p450 | Organism |
|---|---|
| CYP105B1 | Streptomyces griseolus |
| CYP105A1 | Streptomyces griseolus |
| CYP109D1 | Sorangium cellulosum |
| CYP110E1 | Nostoc sp. strain PCC 7120 |
| P450RhF | Rhodococcus sp. NCIMB 9784 |
| CYP102D1 | Streptomyces avermitilis |
| P450cam | Pseudomonas putida |
| CYP102A3 and CYP102A2 | Bacillus subtilis |
| CYP76M8 | Otyza sativa |
| CYP3A4 | Homo sapiens |

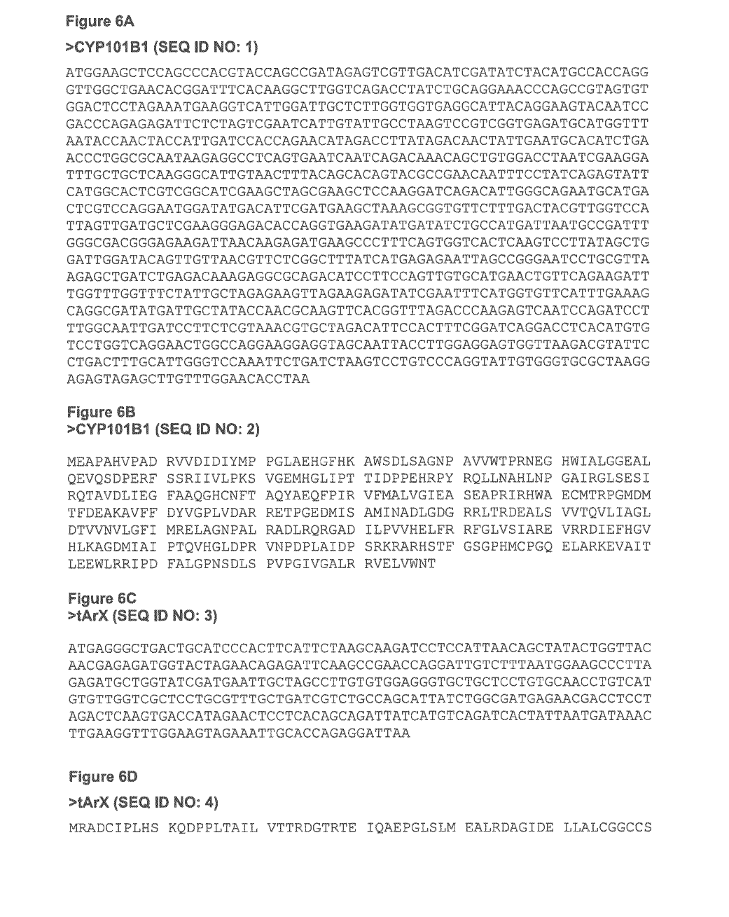

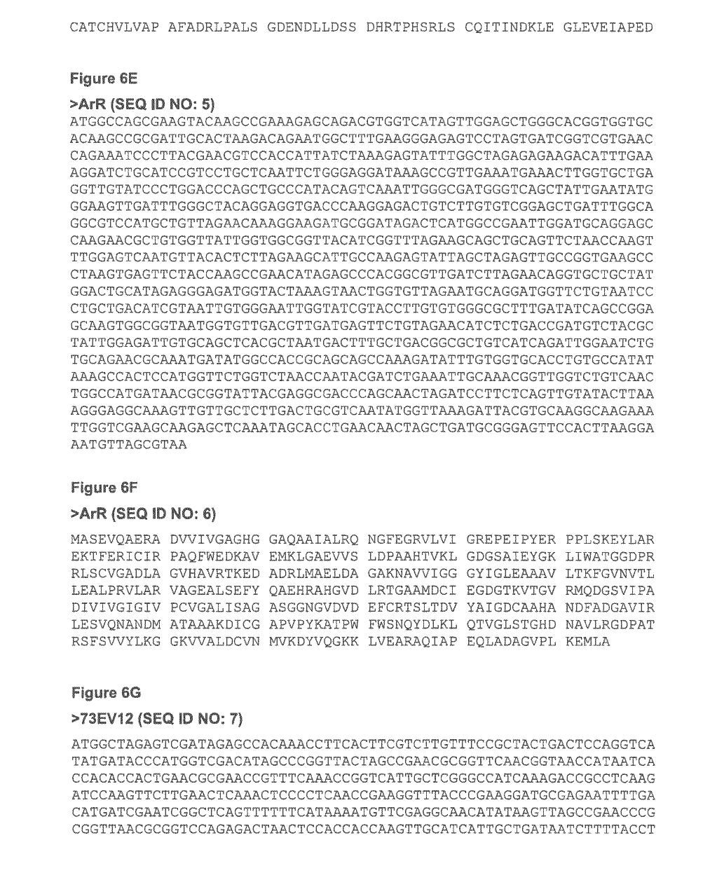

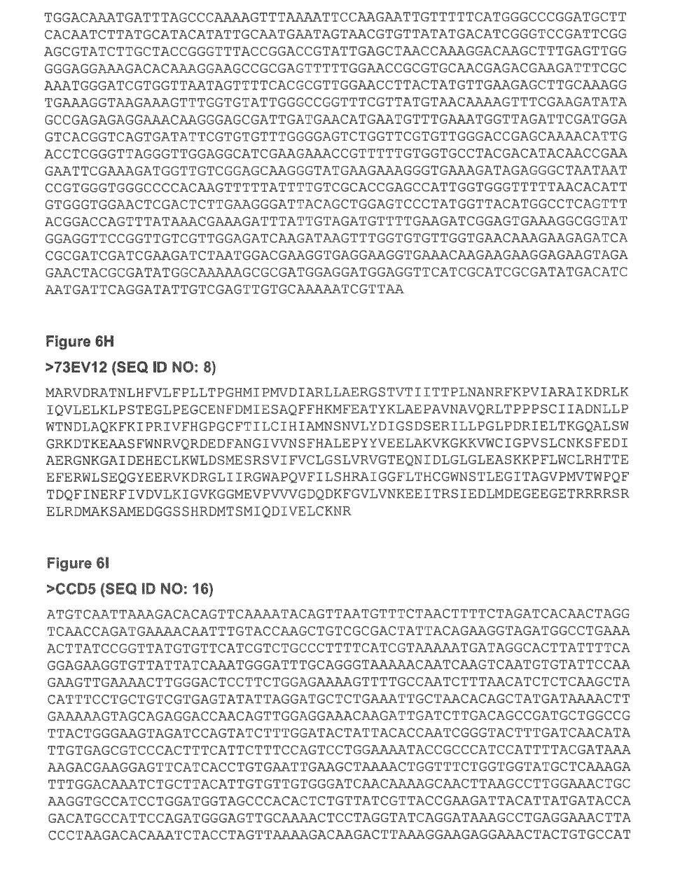

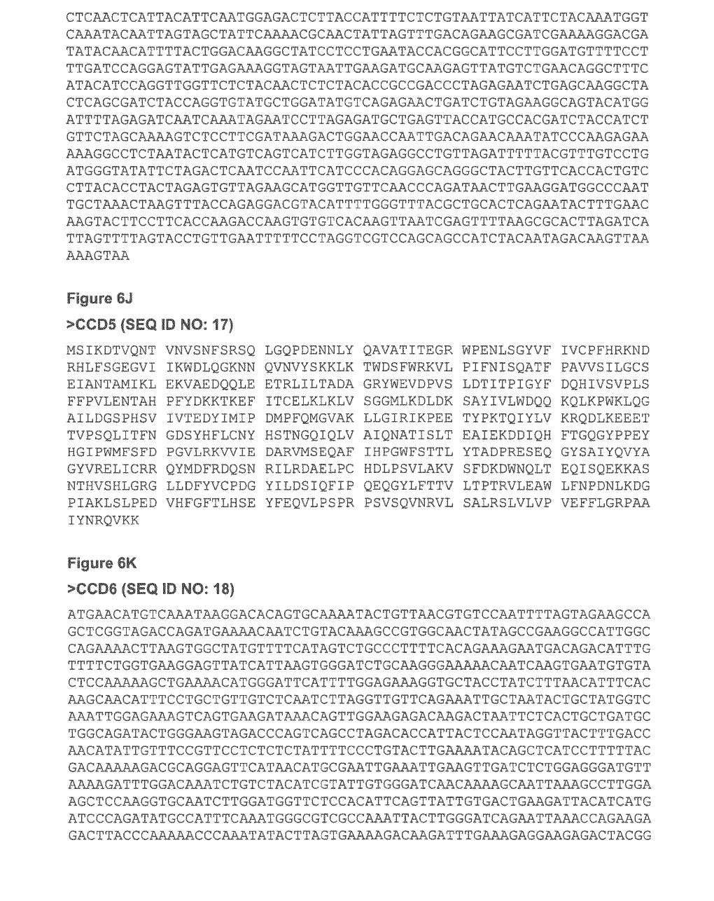

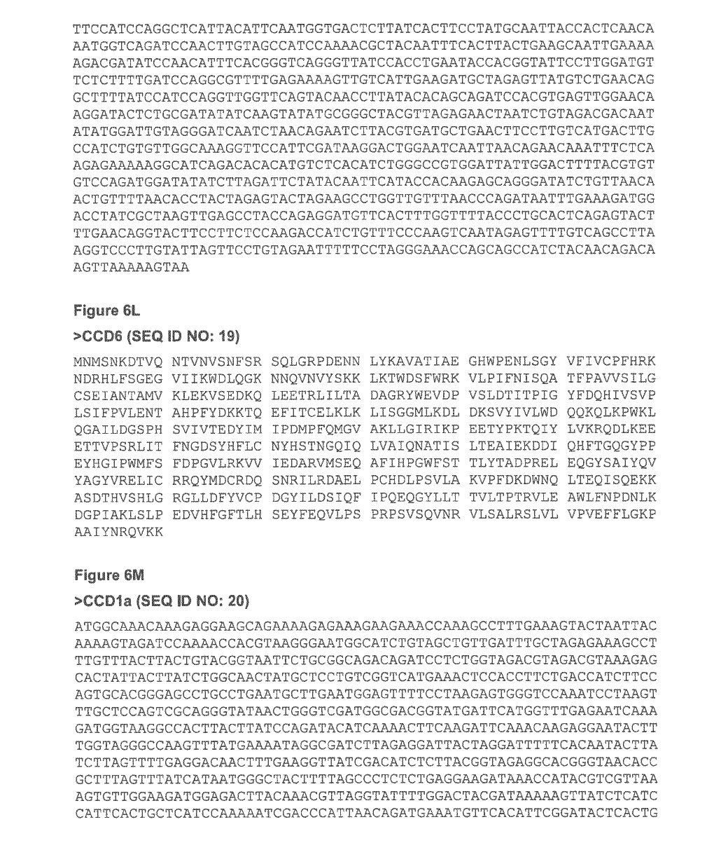

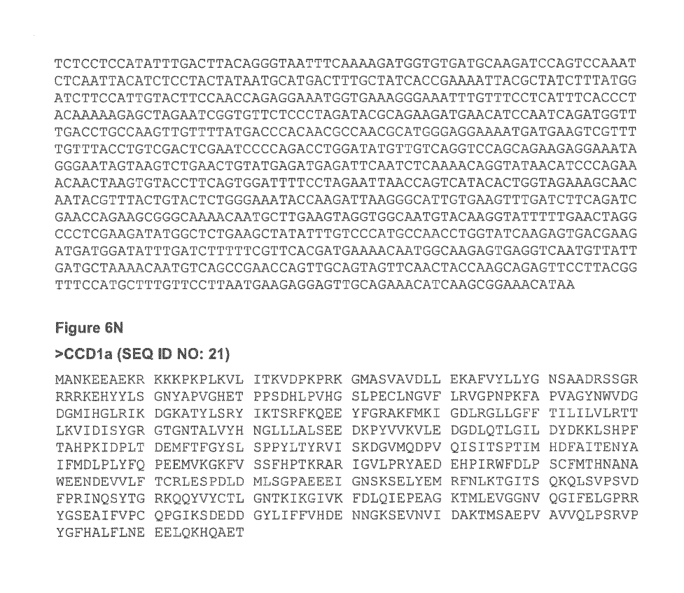

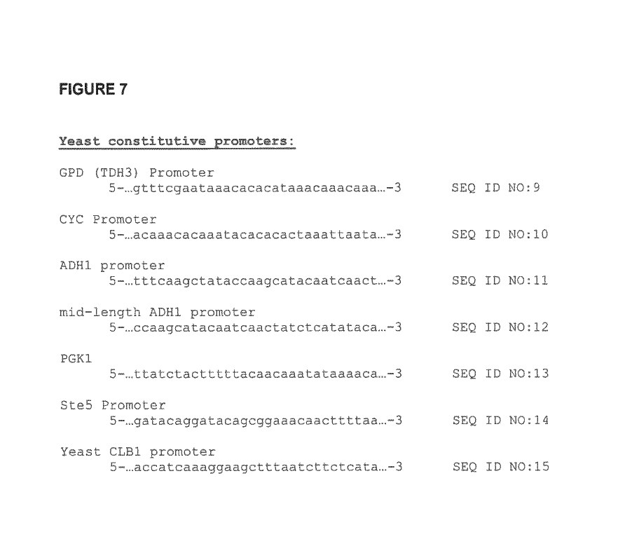

In particular aspects, the cytochrome p450 is Novosphingobium aromaticivorans CYP101B1. The amino acid sequence of CYP101B1 is set forth in FIG. 6.

In some aspects, the ferredoxin is a (2Fe-2S) ferredoxin. In some aspects, the ferredoxin is a truncated (2Fe-2S) ferredoxin. In some aspects, the ferredoxin is truncated Novosphingobium aromaticivorans Arx (tARX). The amino acid sequence of tArX is set forth in FIG. 6.

In some aspects, the flavin-dependent ferredoxin reductase is Novosphingobium aromaticivorans ArR. The amino acid sequence of ArR is set forth in FIG. 6.

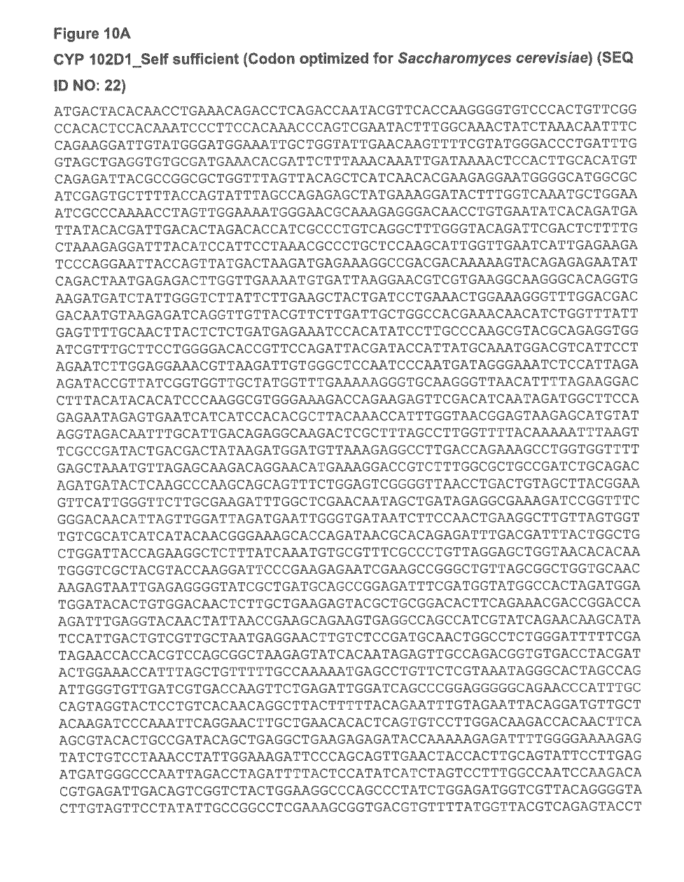

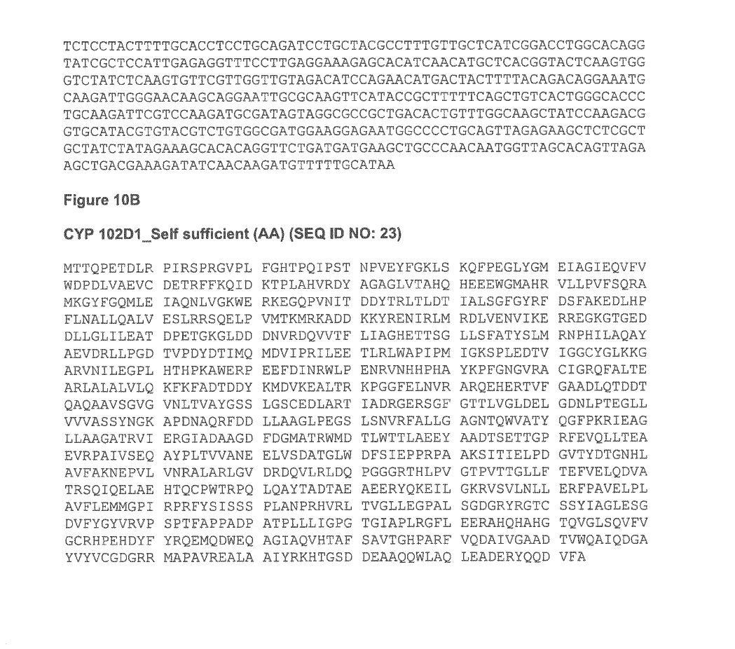

In particular aspects, the cytochrome p450 is Streptomyces avermitilis CYP102D1. The amino acid sequence of CYP102D1 is set forth in FIG. 10.

In particular aspects, the cytochrome p450 disclosed herein (see Table 1) is encoded by a gene construct wherein the gene construct comprises domains from different species. For example, in particular gene constructs the cytochrome p450 domain and the reductase domain are from different endogenous genes or from different recombinant genes or from different species. In some aspects the recombinant host expresses a gene encoding a cytochrome p450 polypeptide wherein the gene encoding the cytochrome p450 polypeptide is set forth in SEQ ID NO: 22, wherein the gene encoding a cytochrome p450 polypeptide is a recombinant gene.

In particular aspects, a recombinant host comprises one or more uridine 5′-diphospho (UDP) glycosyltransferases (UGTs) for the conversion of crocetin to crocin. As used herein, the terms “glycosyltransferases,” “glycosylase enzymes,” or “UGTs” are used interchangeably to refer to any enzyme capable of transferring sugar residues and derivatives thereof (including but not limited to galactose, xylose, rhamnose, glucose, arabinose, glucuronic acid, and others as understood in the art) to acceptor molecules. Acceptor molecules, such as, but not limited to, phenylpropanoids and terpenes include, but are not limited to, other sugars, proteins, lipids and other organic substrates, such as crocetin and crocetin diglucosyl ester. The acceptor molecule can be termed an aglycon (aglucone if the sugar is glucose). An aglycon, includes, but is not limited to, the non-carbohydrate part of a glycoside. Non-limiting examples of UGTs are Stevia rebaudiana 73EV12 and Arabidopsis thaliana UGT85C2.

In particular aspects, expression vectors include, but are not limited to, pETDuet-1 and RSFDuet-1. In some aspects, tArX and ArR are cloned into one vector in tandem. In some aspects, tArX and CYP101B1 are cloned into one vector in tandem. In some aspects, 73EV12 and CYP101B1 are cloned into one vector in tandem. In some aspects, tArX, ArR, CYP101B1 and/or 73EV12 are integrated into genomic DNA of a microorganism host.

In particular aspects, saffron compounds are produced by biosynthesis, biotransformation, or in vitro. In some aspects, the recombinant host cell is cultured in the presence of with β-cyclocitral after recombinant protein expression. In some aspects, the recombinant host cell is cultured in the presence of β-cyclocitral prior to recombinant protein expression.

In some aspects, β-cyclocitral is synthesized in vivo in Saccharomyces cerevisiae, in E. coli, or another heterologous strain via cloning and expression of carotegenic genes responsible for β-carotene production and carotenoid cleavage enzymes such as CODS or CCD6.

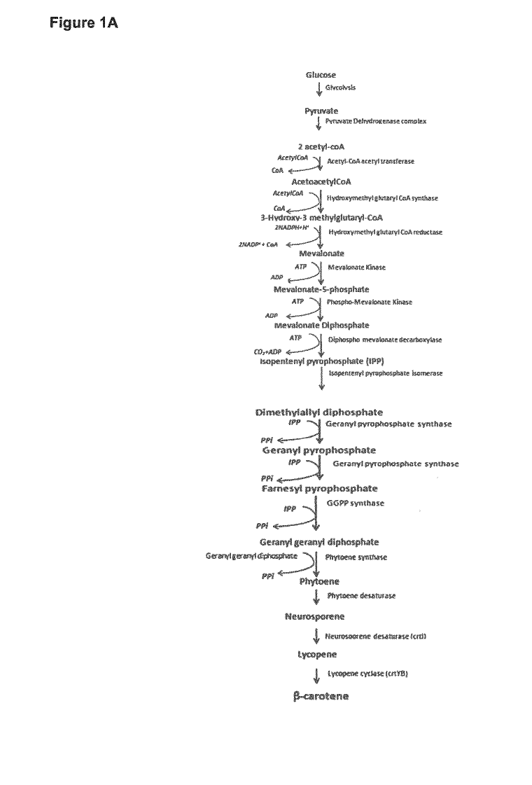

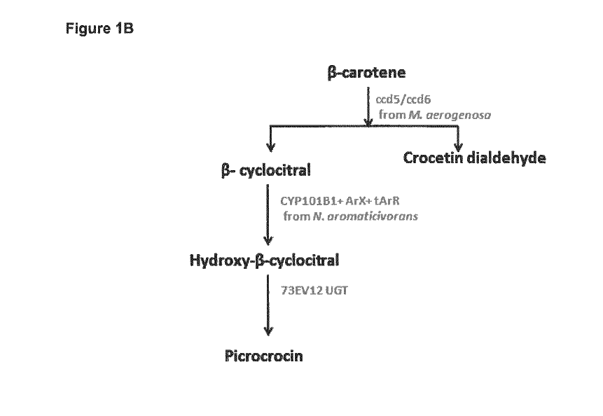

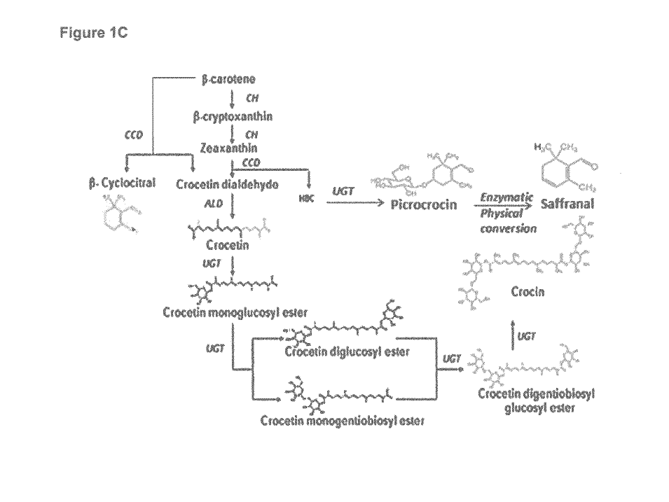

In some aspects, ccd5 is from Microcystis aeroginosa NIES-843 (SEQ ID NO: 16), and ccd6 (SEQ ID NO: 18) is from Microcystis aeroginosa P007806. Ccd5 and ccd6 are cloned into the yeast expression vector YLL055W under a constitutive TPI promoter, and the expression cassette is integrated into the genome of β-carotene producing Saccharomyces cerevisiae. CCD5 (SEQ ID NO: 17) and CCD6 (SEQ ID NO: 19) cleave β-carotene directly into β-cyclocitral and crocetin dialdehyde in a one-step reaction (FIG. 1B). In some aspects, β-cyclocitral is further coverted to hydroxyl-β-cyclocitral with Novosphingobium aromaticivorans CYP101B1, ArX, and tArR. In some aspects, β-cyclocitral is further coverted to hydroxyl-β-cyclocitral with Streptomyces avermitilis CYP102D1. In some aspects, hydroxyl-β-cyclocitral is further converted to picrocrocin with Stevia rebaudiana 73EV12 (FIG. 1B).

In some aspects, hydroxyl-β-cyclocitral is produced from zeaxanthin. In some aspects, β-carotene is hydroxylated into zeaxanthin with a β-carotene hydroxylase, and zeaxanthin is converted into hydroxyl-β-cyclocitral and crocetin dialdehyde (FIG. 10). A non-limiting example of a carotenoid cleavage enzyme that converts zeaxanthin to hydroxyl-β-cyclocitral and crocetin dialdehyde is Crocus sativus CCD1a (SEQ ID NO: 21). In some aspects, hydroxyl-β-cyclocitral is further converted to picrocrocin with Stevia rebaudiana 73EV12.

In some aspects, a glycosylated saffron compound is produced. In some aspects, the glycosylated saffron compound is picrocrocin.

Saffron compounds produced by a recombinant host described herein can be analyzed by techniques generally available to one skilled in the art, for example, but not limited to high-performance liquid chromatography (HPLC) and liquid chromatography-mass spectrometry (LC-MS).

Functional homologs of the polypeptides described above are also suitable for use in producing saffron compounds in a recombinant host. A functional homolog is a polypeptide that has sequence similarity to a reference polypeptide, and that carries out one or more of the biochemical or physiological function(s) of the reference polypeptide. A functional homolog and the reference polypeptide can be natural occurring polypeptides, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional UGT polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide: polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of polypeptides described herein. Sequence analysis can involve BLAST, Reciprocal BLAST, or PSI-BLAST analysis of nonredundant databases using the amino acid sequence of interest as the reference sequence. Amino acid sequence is, in some instances, deduced from the nucleotide sequence. Those polypeptides in the database that have greater than 40% sequence identity are candidates for further evaluation for suitability as polypeptide useful in the synthesis of compounds from saffron. Amino acid sequence similarity allows for conservative amino acid substitutions, such as substitution of one hydrophobic residue for another or substitution of one polar residue for another. When desired, manual inspection of such candidates can be carried out in order to narrow the number of candidates to be further evaluated. Manual inspection can be performed by selecting those candidates that appear to have conserved functional domains.

Conserved regions can be identified by locating a region within the primary amino acid sequence of a polypeptide described herein that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some aspects, alignment of sequences from two different species can be adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some aspects, a conserved region exhibits at least 92%, 94%, 96%, 98%, or 99% amino acid sequence identity.

A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). See Chenna et al., Nucleic Acids Res., 31(13):3497-500 (2003).

ClustalW calculates the best match between a reference and one or more candidate sequences, and aligns them so that identities, similarities, and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that polypeptides described herein can include additional amino acids that are not involved in glucosylation or other enzymatic activities carried out by the enzyme, and thus such a polypeptide can be longer than would otherwise be the case. For example, a polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some aspects, a polypeptide includes an amino acid sequence that functions as a reporter, e.g., a green fluorescent protein or yellow fluorescent protein.

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In some aspects, the coding sequence for a polypeptide described herein is identified in a species other than the recombinant host, i.e., is a heterologous gene. Thus, if the recombinant host is a microorganism, the coding sequence can be from other prokaryotic or eukaryotic microorganisms, from plants or from animals. In some cases, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant gene construct. In addition, stably transformed exogenous genes typically are integrated at positions other than the position where the native sequence is found.

As disclosed herein, a “regulatory region” (prokaryotic and eukaryotic) refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element, or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

One or more genes can be combined in a recombinant nucleic acid construct in “modules” useful for a discrete aspect of production of a compound from saffron. Combining a plurality of genes in a module, particularly a polycistronic module, facilitates the use of the module in a variety of species. For example, tArX, ArR, CYP101B1 and/or 73EV12 can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for production of compounds from saffron, a recombinant construct typically also contains an origin of replication and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

A number of prokaryotes and eukaryotes are suitable for use in constructing the recombinant microorganisms described herein, e.g., gram-negative bacteria, yeast and fungi. A species and strain selected for use as a strain for production of saffron compounds is first analyzed to determine which production genes are endogenous to the strain and which genes are not present (e.g., carotenoid genes). Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species can be suitable. For example, suitable species can be in a genus selected from the group consisting of Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chtysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis and Yarrowia lipolytica. In some aspects, a microorganism can be an Ascomycete such as Gibberella fujikuroi, Kluyveromyces lactis, Schizosaccharomyces pombe, Aspergillus niger, or Saccharomyces cerevisiae. In some aspects, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of compounds from saffron.

Saccharomyces cerevisiae

Saccharomyces cerevisiae is a widely used organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

The genes described herein can be expressed in yeast using any of a number of known promoters. Strains that overproduce terpenes are known and can be used to increase the amount of geranylgeranyl diphosphate available for production of saffron compounds.

In some aspects, auxotrophic markers for cloning include, but are not limited to, HIS3, URA3, TRP1, LEU2, LYS2, ADE2, and GAL, which allow for selection of recombinant strains with an inserted gene of interest. For example, one or more of the auxotrophic markers of strains EY5583-7a (MAT alpha lys2 ADE8 his3 ura3 leu2 trp1) or EFSC 1772 (MAT alpha Δura3 (×2) Δhis3 Δ leu2) can be used during cloning. Auxotrophic markers can be optionally removed from the yeast genome using methods not limited to Ore-Lox recombination or negative selection with 5-fluoroorotic acid (5-FOA). In other aspects, antibiotic resistance, such as kanamycin, can be used as selection marker for construction of recombinant strains.

Suitable strains of S. cerevisiae also can be modified to allow for increased accumulation of storage lipids and/or increased amounts of available precursor molecules by reducing the flow of precursor molecules into competitive pathways. For example, expression of Erg9 can be optionally downregulated, thereby slowing synthesis of ergosterol and upregulating farnesyl pyrophosphate for the synthesis of saffron compounds. In another example, accumulation of triacylglycerols (TAG) up to 30% in S. cerevisiae was demonstrated by Kamisaka et al. (Biochem. J. (2007) 408, 61-68) by disruption of a transcriptional factor SNF2, overexpression of a plant-derived diacyl glycerol acyltransferase 1 (DGA1), and over-expression of yeast LEU2. Furthermore, Froissard et al. (FEMS Yeast Res 9 (2009) 428-438) showed that expression in yeast of AtClo1, a plant oil body-forming protein, will promote oil body formation and result in over-accumulation of storage lipids. Such accumulated TAGs or fatty acids can be diverted towards acetyl-CoA biosynthesis by, for example, further expressing an enzyme known to be able to form acetyl-CoA from TAG (PDX genes) (e.g., a Yarrowia lipolytica PDX gene).

Aspergillus spp.

Aspergillus species such as A. oryzae, A. niger and A. sojae are widely used microorganisms in food production, and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of compounds from saffron.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Agaricus, Gibberella, and Phanerochaete spp.

Agaricus, Gibberella, and Phanerochaete spp. can be useful because they are known to produce large amounts of gibberellin in culture. Thus, the terpene precursors for producing large amounts of compounds from saffron are already produced by endogenous genes. Thus, modules containing recombinant genes for biosynthesis of compounds from saffron can be introduced into species from such genera without further modulating mevalonate or MEP pathway genes.

Rhodobacter spp.

Rhodobacter can be used as the recombinant microorganism platform.

Similar to E. coli, there are libraries of mutants available as well as suitable plasmid vectors, allowing for rational design of various modules to enhance product yield. Isoprenoid pathways have been engineered in membranous bacterial species of Rhodobacter for increased production of carotenoid and CoQ10. See, U.S. Patent Publication Nos. 20050003474 and 20040078846. Methods similar to those described above for E. coli can be used to make recombinant Rhodobacter microorganisms.

Physcomitrella spp.

Physcomitrella mosses, when grown in suspension culture, have characteristics similar to yeast or other fungal cultures. This genera is becoming an important type of cell for production of plant secondary metabolites, which can be difficult to produce in other types of cells.

Plants and Plant Cells

In some aspects, the nucleic acids and polypeptides described herein are introduced into plants or plant cells to produce compounds from saffron. Thus, a host can be a plant or a plant cell that includes at least one recombinant gene described herein. A plant or plant cell can be transformed by having a recombinant gene integrated into its genome, i.e., can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. Seeds produced by a transgenic plant can be grown and undergo self-fertilization (fusion of gametes from the same plant) to obtain seeds homozygous for the nucleic acid construct. Conversely, the seeds produced by a transgenic plant can be grown, and the progeny can be outcrossed (gametes fused from different plants) and subsequently self-fertilized to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

A population of transgenic plants can be screened and/or selected for those members of the population that have a trait or phenotype conferred by expression of the transgene. For example, a population of progeny of a single transformation event can be screened for those plants having a desired level of expression of a tArX, ArR, CYP101B1, CYP102D1, or 73EV12 polypeptide or nucleic acid. Physical and biochemical methods can be used to identify expression levels. These include Southern analysis or PCR amplification for detection of a polynucleotide; Northern blots, 51 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or nucleic acids. Methods for performing all of the referenced techniques are known. As an alternative, a population of plants comprising independent transformation events can be screened for those plants having a desired trait, such as production of a compound from saffron. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in a level of a saffron compound relative to a control plant that lacks the transgene.

The nucleic acids, recombinant genes, and constructs described herein can be used to transform a number of monocotyledonous and dicotyledonous plants and plant cell systems. Non-limiting examples of suitable monocots include, for example, cereal crops such as rice, rye, sorghum, millet, wheat, maize, and barley. The plant also can be a dicot such as soybean, cotton, sunflower, pea, geranium, spinach, or tobacco. In some cases, the plant can contain the precursor pathways for phenyl phosphate production such as the mevalonate pathway, typically found in the cytoplasm and mitochondria. The non-mevalonate pathway is more often found in plant plastids [Dubey, et al., 2003 J. Biosci. 28 637-646]. One with skill in the art can target expression of biosynthesis polypeptides to the appropriate organelle through the use of leader sequences, such that biosynthesis occurs in the desired location of the plant cell. One with skill in the art will use appropriate promoters to direct synthesis, e.g., to the leaf of a plant, if so desired. Expression can also occur in tissue cultures such as callus culture or hairy root culture, if so desired.

The invention will be further described in the following examples, which do not limit the scope of the invention described in the claims.

### EXAMPLES

The Examples that follow are illustrative of specific embodiments of the invention, and various uses thereof. They are set forth for explanatory purposes only and are not to be taken as limiting the invention.

### Example 1

### Biosynthesis of Hydroxy-β-Cyclocitral from β-Cyclocitral Using Cytochrome P450, CYP101B1

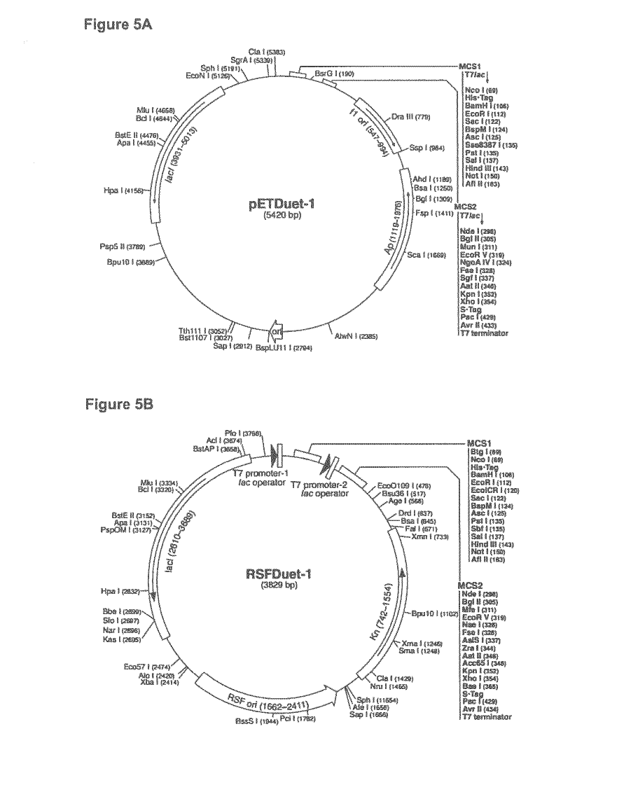

A previously undisclosed route of hydroxy-β-cyclocitral synthesis in recombinant cells is disclosed herein, wherein β-cyclocitral is hydroxylated using a cytochrome P450 class I electron transfer system comprising CYP101B1 (cytochrome p450), ArR (flavin-dependent ferredoxin reductase), and tArX (truncated [2Fe-2S] ferredoxin). All genes were from Novosphingobium aromaticivorans. In the first construct, tArX (SEQ ID NO: 3) and ArR (SEQ ID NO: 5) were cloned into the pETDuet-1 vector. The tArX gene was PCR amplified and inserted into the pETDuet-1 (FIG. 5A) vector using the NcoI and HindIII restriction sites. The ArR gene was then PCR amplified and inserted using the NdeI and KpnI restriction sites. In a second construct, tArX (SEQ ID NO: 3) and CYP101B1 (SEQ ID NO: 1) were cloned into the pRSFDuet-1 (FIG. 5B) vector. The tArX gene was inserted using the NcoI and HindIII restriction sites, and CYP101B1 was subsequently inserted using the NdeI and EcoRV restriction sites.

The tArX-ArR-pETDuet-1 and tArX-CYP101B1-pRSFDuet-1 plasmids were co-transformed and expressed in the E. coli BL21-Gold (DE3) pLysS strain (Stratagene) to provide in vivo whole-cell substrate oxidation systems. The recombinant E. coli DE3 cells were grown in 2×YT broth to an OD₆₀₀ of 0.6 and induced with 60 μM IPTG and 0.8 mM δ-amino levulinic acid. The culture was incubated in shaker flask culture at 20° C. and 110 rpm for 24 h. The cells were pelleted by centrifugation at 25° C. and 4000 rpm for 10 min. 2 mL of E. coli minimal media with ampicillin and kanamycin (EMM) as well as 2 mM β-cyclocitral were added to the pellet; the culture was incubated at 20° C. and 220 rpm overnight.

The pellet was resuspended in 100% w/v HPLC grade methanol and incubated at −18° C. overnight. The sample was then centrifuged at 10000 rpm for 2 min. Formation of hydroxy-β-cyclocitral was measured via HPLC and GCMS analysis of the supernatant. HPLC analysis was performed with a Shimadzu LC 8A system equipped with a Shimadzu SPD M20A PDA (Photo Diode Array) Detector and fitted with a Gemini NX 018 column (25 cm×4.6 mm). The mobile phase was acetonitrile:H₂O (a linear gradient of 20% acetonitrile to 80% acetonitrile over a period of 20 min followed by 100% Acetonitrile for 5 min). For detection, scanning was done from 190 nm to 800 nm, where peaks at 250 nm correspond to hydroxyl-β-cyclocitral.

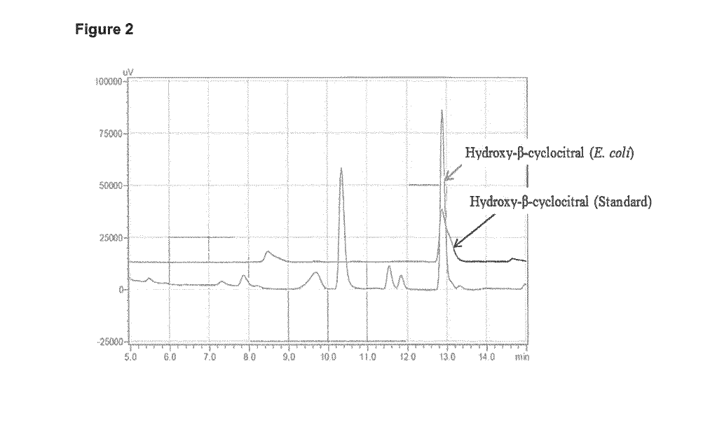

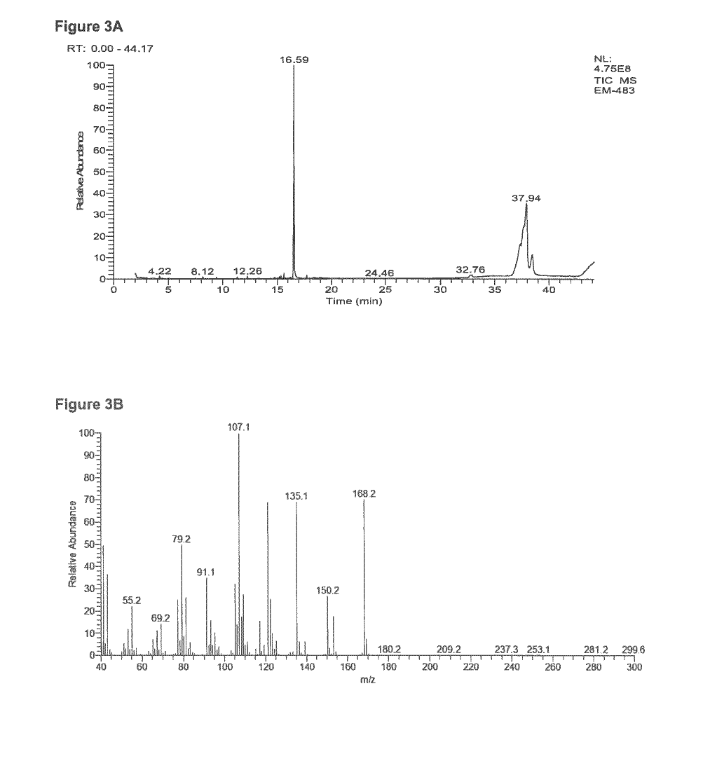

GCMS analysis was performed using Thermo GC-Trace ultra VER 5.0, Thermo MS DSQ II equipment fitted with a DB 35-MS capillary standard non-polar column (30 MTS, ID-0.25 MM, Film-0.25 microM). The following conditions were used: helium carrier gas at a flow rate of 1.0 mL/min; 70° C. raised to 250° C. at 10 min hold. The HPLC and GCMS spectra showing hydroxyl-β-cyclocitral formation in the described E. coli culture are shown in FIGS. 2 and 3, respectively.

### Example 2

### Biosynthesis of Picrocrocin Using the p450 Class I Transfer System and UGT 73EV12

Picrocrocin was produced from β-carotene, as shown in FIG. 3. As described in Example 1, tArX (SEQ ID NO: 3) and ArR (SEQ ID NO: 5) were cloned into the pETDuet-1 vector. In a separate construct, the UGT 73EV12 (SEQ ID NO: 7) and CYP01B1 (SEQ ID NO: 1) genes were cloned into the pRSFDuet-1 vector using the SacI/SbfI and NdeI/EcoRV restriction sites, respectively.

The tArX-ArR-pETDuet-1 and 73EV12UGT-CYP101B1-pRSFDuet-1 plasmids were co-transformed and expressed in the E. coli BL21-Gold (DE3) pLysS strain. The recombinant E. coli DE3 cells were grown in 2× YT broth to an OD₆₀₀ of 0.6 and induced with 60 μM IPTG and 0.8 mM δ-amino levulinic acid. The culture was incubated in shaker flask culture at 20° C. and 110 rpm for 24 h. The cells were pelleted by centrifugation at 25° C. and 4000 rpm for 10 min. 2 mL of E. coli minimal media with ampicillin and kanamycin (EMM) as well as 2 mM β-cyclocitral were added to the pellet; the culture was incubated at 20° C. and 220 rpm overnight.

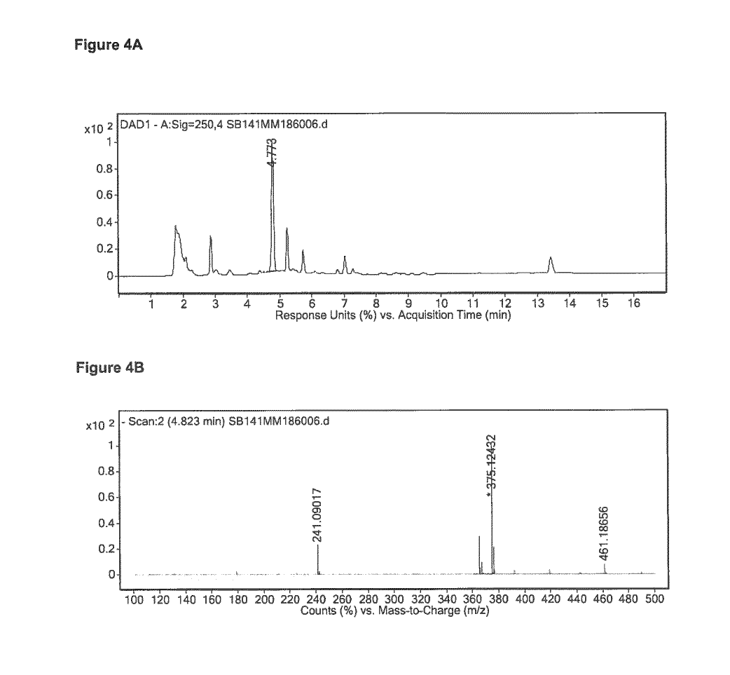

The pellet was resuspended in 100% w/v HPLC grade methanol and incubated at −18° C. overnight. The sample was then centrifuged at 10000 rpm for 2 min. The culture sample was analyzed for the formation of picrocrocin by LCMS. An Agilent 6520 Quadrupole time-of-flight (Q-TOF) mass spectrometer (G6510A) coupled to an Agilent 1200 series RRLC system was used. The separation was carried out on a reverse-phase Gemini 018 column (4.6×100 mm, 110° A) at ambient temperature. Step gradient elution was employed using 0.1% formic acid in water (solvent A) and acetonitrile (% acetonitrile: 10, 25, 80, 80, 10) with a flow rate of 0.8 mL/min, a run time of 22 min, and a post-run time 5 min). Sample detection was carried out at 250 nm for picrocrocin using a UV detector. For MS analysis, the Agilent's Q-TOF mass spectrometer was equipped with a dual ESI ion source. Mass spectra were acquired using the fast polar switching mode with a scan range from m/z 100 to 600 Da and a scan rate of 1.01 by using reference masses enabled mode with average scans 1/s. The conditions of dual ESI source were as followed: drying gas (N₂) flow rate: 10.0 l/min; temperature: 325° C.; pressure of nebulizer: 60 psi; capillary voltage: 3500V, Vcap-3500, Fragmentor-175, and Skimme-65 and OctopoleRFPeak 750. Data were acquired and analyzed by Agilent Mass Hunter Workstation Software version B.02.01 (B2116.20) (Agilent Technologies). The output signal was monitored and processed using mass hunter software on Intel® Core™ 2 Duo computer (HP xw 4600 Workstation). The resulting LCMS spectrum is shown in FIG. 4.

### Example 3

### Use of a 3-Cyclocitral Producing Yeast Strain for the Biosynthesis of Hydroxy-3-Cyclocitral from 3-Cyclocitral

A beta cyclocitral producing yeast strain was created using standard molecular biology protocols (see Table 2 below). Multiple gene copies were integrated into the genome of yeast so that enough beta cyclocitral was produced in the strain for acting as substrate.

TABLE 2

| | | | | | Integration |
|---|---|---|---|---|---|
| | | | | | site |
| S. NO | Vector | Promoter | Genes | Terminator | Involved |
| 1 | ECM3 | GPD, pTPI | crtYB, crtE, Nc-crtl | CYC, tTPI | ECM3, KIN1 |
| 2 | EPSB2549 | GPD, pTPI | CCD6, Ald9 | CYC, tTPI | EXG1 KO |
| 3 | YLL | GPD, pTPI | CCD6, Ald9 | CYC, tTPI | YLL055w-X11 |
| 4 | PRP5 | GPD, pTPI, PGK1, TEF1 | UGT17 (UN1671), 75L6, | CYC, tTPI, tTDH2, | PRP5-II |
| | | | CCD6 | tFBA | |
| 6 | EPSB508 | GPD, pTPI | UGT17 (UN1671), 75L6 | CYC, tTPI | X115 |

Combination of CrtI, CrtYB and CrtE produces beta carotene which is then cleaved by CCD6 to form crocetin dialdehdye and beta cyclocitral. Crocetin dialdehdye is then oxidized to crocetin by ALD9 and subsequently glycosylated to crocin by UGT75L6 and UGT1671. Beta cyclocitral biosynthesized in this strain will act as a substrate for CYP for HBC biosynthesis. Glycosylation of HBC leads to formation of Picrocrocin which is catalysed by UGT73EV12.

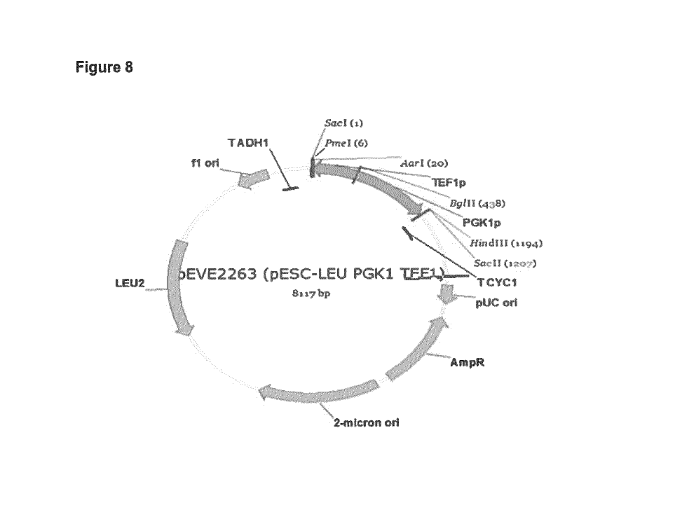

A previously undisclosed route of hydroxy-β-cyclocitral synthesis in recombinant cells is disclosed herein, wherein β-cyclocitral is hydroxylated using a cytochrome P450 comprising CYP102D1 from Streptomyces avermitilis. The CYP102D gene was synthesized and codon optimized for Saccharomyces cerevisiae. Specifically CYP102D1 gene was cloned in a yeast dual expression vector named pEVE2263 (FIG. 8) under a PGK1 promoter using Hind III and SacII restriction sites. The gycosyltransferase gene 73EV12 gene was cloned under a TEF1 promoter using PmeI and AarI sites. The recombinant pEVE2263 expression vector harbouring CYP102D1 and 73EV12 was transformed into the 20 yeast strain. The positive clones were screened by analytical PCR and sequencing of the recombinant plasmid. The recombinant S. cerevisiae cells were grown in 20% glucose containing SC-drop out media lacking leucine for 72 hrs at 30° C. (250 rpm).

The yeast culture was thereafter pelleted at 13,000 rpm for 10 minutes and supernatant collected was used for extraction and analysis of picrocrocin production by HPLC. Each yeast cell culture supernatant was taken (5 ml), dried under freezing condition, suspended in minimum volume of methanol: water 1:1 and precipitated with 1:0.5 v/v of acetonitrile and methanol:water at −18° C. overnight, centrifuged at 10000 rpm for 2 min and injected in HPLC for the detection of hydroxy-β-cyclocitral, picrocrocin, and 6-cyclocitral.

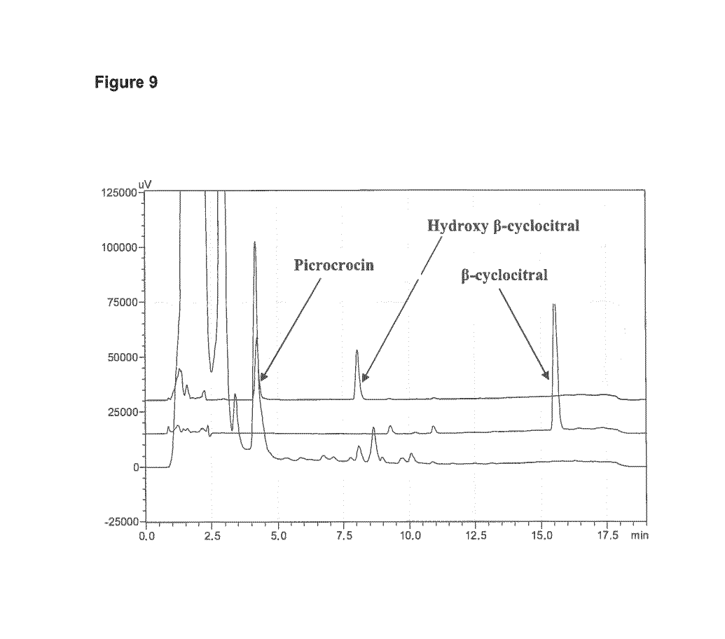

HPLC analysis was performed with a Shimadzu LC 8A system equipped with a Shimadzu SPD M20A PDA (Photo Diode Array) Detector and fitted with a Phenomenex 018 LUNA (15 cm length×4.6 mm i.d) 5 μm particle size. The mobile phase was Acetonitrile:Water (A linear gradient of 20% Acetonitrile to 80% Acetonitrile over a period of 10 minutes, then 80 to 100% acetonitrile for the next 2 minutes). For detection, scanning was done from 190 nm to 800 nm, where peaks at 250 nm correspond to hydroxyl-β-cyclocitral and Picrocrocin. The HPLC spectra showing hydroxy-β-cyclocitral and picrocrocin formation in the described 20 yeast strains is shown in FIG. 9.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A recombinant host that expresses a gene encoding a cytochrome p450 polypeptide, a gene encoding a (2Fe-2S) ferredoxin polypeptide, and a gene encoding a flavin-dependent ferredoxin reductase, wherein at least one of said genes is a recombinant gene.

2. The recombinant host of claim 1, wherein the cytochrome p450 polypeptide comprises a cytochrome p450 polypeptide having 40% or greater identity to the amino acid sequence set forth in SEQ ID NO:2.

3. The recombinant host of claim 1, wherein the (2Fe-2S) ferredoxin polypeptide comprises a (2Fe-2S) ferredoxin polypeptide having 40% or greater identity to the amino acid sequence set forth in SEQ ID NO:4.

4. The recombinant host of claim 1, wherein the flavin-dependent ferredoxin reductase polypeptide comprises a flavin-dependent ferredoxin reductase polypeptide having 40% or greater identity to the amino acid sequence set forth in SEQ ID NO:6.

5. The recombinant host of claim 1, wherein the cell further expresses a gene encoding a uridine 5′-diphospho glycosyltransferase (UGT) polypeptide.

6. The recombinant host of claim 5, wherein the gene is set forth in SEQ ID NO:7.

7. The host of claim 5, wherein the UGT polypeptide comprises a polypeptide having 40% or greater identity to the amino acid sequence set forth in SEQ ID NO:8.

8. The recombinant host of any one of claims 1-7, herein the recombinant host comprises a yeast cell, a plant cell, a mammalian cell, an insect cell, a fungal cell, a bacterial cell, an algal cell, or a cyanobacterial cell.

9. The recombinant host of claim 8, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

10. The recombinant host of claim 8, wherein the yeast cell is a Saccharomycete.

11. The recombinant host of claim 10, wherein the yeast cell is a Saccharomyces cerevisiae cell.

12. The recombinant host of any one of claims 1-4 and 8-11, wherein the cell produces hydroxy-β-cyclocitral.

13. The recombinant host any one of claims 5-11, wherein the cell produces picrocrocin.

14. A method of producing a saffron compound, comprising cultivating the recombinant host of any one of claim 1-4, or 8-11 in a culture medium under conditions in which the gene encoding a cytochrome p450 polypeptide, the gene encoding a (2Fe-2S) ferredoxin polypeptide, and the gene encoding a flavin-dependent ferredoxin reductase are expressed, wherein the saffron compound is hydroxyl-β-cyclocitral.

15. The method of claim 14, wherein the gene encoding a cytochrome p450 polypeptide has an amino acid sequence set forth in SEQ ID NO:2, the gene encoding a (2Fe-2S) ferredoxin polypeptide has an amino acid sequence set forth in SEQ ID NO:4, and the gene encoding a flavin-dependent ferredoxin reductase polypeptide has an amino acid sequence set forth in SEQ ID NO:6.

16. A method of producing a saffron compound, comprising cultivating the recombinant host of any one of claims 5-11 in a culture medium under conditions in which the gene encoding a cytochrome p450 polypeptide, the gene encoding a (2Fe-2S) ferredoxin polypeptide, the gene encoding a flavin-dependent ferredoxin reductase, and the gene encoding UGT polypeptide are expressed, wherein the saffron compound is a glycosylated saffron compound, wherein the glycosylated saffron compound is picrocrocin.

17. The method of claim 16, wherein the gene encoding a cytochrome p450 polypeptide has an amino acid sequence set forth in SEQ ID NO:2, the gene encoding a (2Fe-2S) ferredoxin gene has an amino acid sequence set forth in SEQ ID NO:4, the gene encoding a flavin-dependent ferredoxin reductase polypeptide has an amino acid sequence set forth in SEQ ID NO: 6, and the gene encoding a UGT polypeptide has an amino acid sequence set forth in SEQ ID NO:8.

18. The method of any one of claims 15-17, wherein the recombinant host is a yeast cell, a plant cell, a mammalian cell, an insect cell, a fungal cell, or a bacterial cell.

19. The method of claim 18, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

20. The method of claim 18, wherein the yeast cell is a Saccharomycete.

21. The method of claim 20, wherein the yeast cell is a Saccharomyces cerevisiae cell.

22. A recombinant host that expresses a gene encoding a cytochrome p450 polypeptide wherein the gene encoding the cytochrome p450 polypeptide is set forth in SEQ ID NO: 22.

23. The recombinant host of claim 22, wherein the cytochrome p450 polypeptide comprises a cytochrome p450 polypeptide having 40% or greater identity to the amino acid sequence set forth in SEQ ID NO: 23.

24. The recombinant host claim 22, wherein the cell further expresses a gene encoding a uridine 5′-diphospho glycosyltransferase (UGT) polypeptide.

25. The recombinant host claim 24, wherein the gene is set forth in SEQ ID NO:7.

26. The cell of claim 24, wherein the UGT polypeptide comprises a polypeptide having 40% or greater identity to the amino acid sequence set forth in SEQ ID NO:8.

27. The recombinant host of any one of claims 22-24, wherein the recombinant host comprises a yeast cell, a plant cell, a mammalian cell, an insect cell, a fungal cell, a bacterial cell, an algal cell, or a cyanobacterial cell.

28. The recombinant host of claim 27, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

29. The recombinant host of claim 27, wherein the yeast cell is a Saccharomycete.

30. The recombinant host of claim 29, wherein the yeast cell is a Saccharomyces cerevisiae cell.

31. The recombinant host of any one of claims 22-23 and 27-30, wherein the cell produces hydroxy-β-cyclocitral.

32. The recombinant host of any one of claims 24-30, wherein the cell produces picrocrocin.

33. A method of producing a saffron compound, comprising cultivating the recombinant host of any one of claims 22-23 and 27-31 in a culture medium under conditions in which the gene encoding a cytochrome p450 polypeptide is expressed, wherein the saffron compound is hydroxyl-β-cyclocitral.

34. A method of producing a saffron compound, comprising cultivating the recombinant host of any one of claims 24-30 in a culture medium under conditions in which the gene encoding a cytochrome p450 polypeptide and the gene encoding UGT polypeptide are expressed, wherein the saffron compound is a glycosylated saffron compound, wherein the glycosylated saffron compound is picrocrocin.

35. The method of any one of claims 33-34, wherein the recombinant host comprises a yeast cell, a plant cell, a mammalian cell, an insect cell, a fungal cell, or a bacterial cell.

36. The method of claim 35, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

37. The method of claim 35, wherein the yeast cell is a Saccharomycete.

38. The method of claim 37, wherein the yeast cell is a Saccharomyces cerevisiae cell.


SEQUENCE LISTING (23 sequences)

SEQ ID NO: 1 - DNA, 1197 bp, Novosphingobium aromaticivorans
atggaagctccagcccacgtaccagccgatagagtcgttgacatcgatatctacatgccaccagggttggctgaacacggatttcacaaggcttggtcagacctatctgcaggaaacccagccgtagtgtggactcctagaaatgaaggtcattggattgctcttggtggtgaggcattacaggaagtacaatccgacccagagagattctctagtcgaatcattgtattgcctaagtccgtcggtgagatgcatggtttaataccaactaccattgatccaccagaacatagaccttatagacaactattgaatgcacatctgaaccctggcgcaataagaggcctcagtgaatcaatcagacaaacagctgtggacctaatcgaaggatttgctgctcaagggcattgtaactttacagcacagtacgccgaacaatttcctatcagagtattcatggcactcgtcggcatcgaagctagcgaagctccaaggatcagacattgggcagaatgcatgactcgtccaggaatggatatgacattcgatgaagctaaagcggtgttctttgactacgttggtccattagttgatgctcgaagggagacaccaggtgaagatatgatatctgccatgattaatgccgatttgggcgacgggagaagattaacaagagatgaagccctttcagtggtcactcaagtccttatagctggattggatacagttgttaacgttctcggctttatcatgagagaattagccgggaatcctgcgttaagagctgatctgagacaaagaggcgcagacatccttccagttgtgcatgaactgttcagaagatttggtttggtttctattgctagagaagttagaagagatatcgaatttcatggtgttcatttgaaagcaggcgatatgattgctataccaacgcaagttcacggtttagacccaagagtcaatccagatcctttggcaattgatccttctcgtaaacgtgctagacattccactttcggatcaggacctcacatgtgtcctggtcaggaactggccaggaaggaggtagcaattaccttggaggagtggttaagacgtattcctgactttgcattgggtccaaattctgatctaagtcctgtcccaggtattgtgggtgcgctaaggagagtagagcttgtttggaacacctaa

SEQ ID NO: 2 - protein, 398 aa, Novosphingobium aromaticivorans
MEAPAHVPADRVVDIDIYMPPGLAEHGFHKAWSDLSAGNPAVVWTPRNEGHWIALGGEALQEVQSDPERFSSRIIVLPKSVGEMHGLIPTTIDPPEHRPYRQLLNAHLNPGAIRGLSESIRQTAVDLIEGFAAQGHCNFTAQYAEQFPIRVFMALVGIEASEAPRIRHWAECMTRPGMDMTFDEAKAVFFDYVGPLVDARRETPGEDMISAMINADLGDGRRLTRDEALSVVTQVLIAGLDTVVNVLGFIMRELAGNPALRADLRQRGADILPVVHELFRRFGLVSIAREVRRDIEFHGVHLKAGDMIAIPTQVHGLDPRVNPDPLAIDPSRKRARHSTFGSGPHMCPGQELARKEVAITLEEWLRRIPDFALGPNSDLSPVPGIVGALRRVELVWNT

SEQ ID NO: 3 - DNA, 363 bp, Novosphingobium aromaticivorans
atgagggctgactgcatcccacttcattctaagcaagatcctccattaacagctatactggttacaacgagagatggtactagaacagagattcaagccgaaccaggattgtctttaatggaagcccttagagatgctggtatcgatgaattgctagccttgtgtggagggtgctgctcctgtgcaacctgtcatgtgttggtcgctcctgcgtttgctgatcgtctgccagcattatctggcgatgagaacgacctcctagactcaagtgaccatagaactcctcacagcagattatcatgtcagatcactattaatgataaacttgaaggtttggaagtagaaattgcaccagaggattaa

SEQ ID NO: 4 - protein, 120 aa, Novosphingobium aromaticivorans
MRADCIPLHSKQDPPLTAILVTTRDGTRTEIQAEPGLSLMEALRDAGIDELLALCGGCCSCATCHVLVAPAFADRLPALSGDENDLLDSSDHRTPHSRLSCQITINDKLEGLEVEIAPED

SEQ ID NO: 5 - DNA, 1248 bp, Novosphingobium aromaticivorans
atggccagcgaagtacaagccgaaagagcagacgtggtcatagttggagctgggcacggtggtgcacaagccgcgattgcactaagacagaatggctttgaagggagagtcctagtgatcggtcgtgaaccagaaatcccttacgaacgtccaccattatctaaagagtatttggctagagagaagacatttgaaaggatctgcatccgtcctgctcaattctgggaggataaagccgttgaaatgaaacttggtgctgaggttgtatccctggacccagctgcccatacagtcaaattgggcgatgggtcagctattgaatatgggaagttgatttgggctacaggaggtgacccaaggagactgtcttgtgtcggagctgatttggcaggcgtccatgctgttagaacaaaggaagatgcggatagactcatggccgaattggatgcaggagccaagaacgctgtggttattggtggcggttacatcggtttagaagcagctgcagttctaaccaagtttggagtcaatgttacactcttagaagcattgccaagagtattagctagagttgccggtgaagccctaagtgagttctaccaagccgaacatagagcccacggcgttgatcttagaacaggtgctgctatggactgcatagagggagatggtactaaagtaactggtgttagaatgcaggatggttctgtaatccctgctgacatcgtaattgtgggaattggtatcgtaccttgtgtgggcgctttgatatcagccggagcaagtggcggtaatggtgttgacgttgatgagttctgtagaacatctctgaccgatgtctacgctattggagattgtgcagctcacgctaatgactttgctgacggcgctgtcatcagattggaatctgtgcagaacgcaaatgatatggccaccgcagcagccaaagatatttgtggtgcacctgtgccatataaagccactccatggttctggtctaaccaatacgatctgaaattgcaaacggttggtctgtcaactggccatgataacgcggtattacgaggcgacccagcaactagatccttctcagttgtatacttaaagggaggcaaagttgttgctcttgactgcgtcaatatggttaaagattacgtgcaaggcaagaaattggtcgaagcaagagctcaaatagcacctgaacaactagctgatgcgggagttccacttaaggaaatgttagcgtaa

SEQ ID NO: 6 - protein, 415 aa, Novosphingobium aromaticivorans
MASEVQAERADVVIVGAGHGGAQAAIALRQNGFEGRVLVIGREPEIPYERPPLSKEYLAREKTFERICIRPAQFWEDKAVEMKLGAEVVSLDPAAHTVKLGDGSAIEYGKLIWATGGDPRRLSCVGADLAGVHAVRTKEDADRLMAELDAGAKNAVVIGGGYIGLEAAAVLTKFGVNVTLLEALPRVLARVAGEALSEFYQAEHRAHGVDLRTGAAMDCIEGDGTKVTGVRMQDGSVIPADIVIVGIGIVPCVGALISAGASGGNGVDVDEFCRTSLTDVYAIGDCAAHANDFADGAVIRLESVQNANDMATAAAKDICGAPVPYKATPWFWSNQYDLKLQTVGLSTGHDNAVLRGDPATRSFSVVYLKGGKVVALDCVNMVKDYVQGKKLVEARAQIAPEQLADAGVPLKEMLA

SEQ ID NO: 7 - DNA, 1470 bp, Stevia rebaudiana
atggctagagtcgatagagccacaaaccttcacttcgtcttgtttccgctactgactccaggtcatatgatacccatggtcgacatagcccggttactagccgaacgcggttcaacggtaaccataatcaccacaccactgaacgcgaaccgtttcaaaccggtcattgctcgggccatcaaagaccgcctcaagatccaagttcttgaactcaaactcccctcaaccgaaggtttacccgaaggatgcgagaattttgacatgatcgaatcggctcagttttttcataaaatgttcgaggcaacatataagttagccgaacccgcggttaacgcggtccagagactaactccaccaccaagttgcatcattgctgataatcttttaccttggacaaatgatttagcccaaaagtttaaaattccaagaattgtttttcatgggcccggatgcttcacaatcttatgcatacatattgcaatgaatagtaacgtgttatatgacatcgggtccgattcggagcgtatcttgctaccgggtttaccggaccgtattgagctaaccaaaggacaagctttgagttgggggaggaaagacacaaaggaagccgcgagtttttggaaccgcgtgcaacgagacgaagatttcgcaaatgggatcgtggttaatagttttcacgcgttggaaccttactatgttgaagagcttgcaaaggtgaaaggtaagaaagtttggtgtattgggccggtttcgttatgtaacaaaagtttcgaagatatagccgagagaggaaacaagggagcgattgatgaacatgaatgtttgaaatggttagattcgatggagtcacggtcagtgatattcgtgtgtttggggagtctggttcgtgttgggaccgagcaaaacattgacctcgggttagggttggaggcatcgaagaaaccgtttttgtggtgcctacgacatacaaccgaagaattcgaaagatggttgtcggagcaagggtatgaagaaagggtgaaagatagagggctaataatccgtgggtgggccccacaagtttttattttgtcgcaccgagccattggtgggtttttaacacattgtgggtggaactcgactcttgaagggattacagctggagtccctatggttacatggcctcagtttacggaccagtttataaacgaaagatttattgtagatgttttgaagatcggagtgaaaggcggtatggaggttccggttgtcgttggagatcaagataagtttggtgtgttggtgaacaaagaagagatcacgcgatcgatcgaagatctaatggacgaaggtgaggaaggtgaaacaagaagaaggagaagtagagaactacgcgatatggcaaaaagcgcgatggaggatggaggttcatcgcatcgcgatatgacatcaatgattcaggatattgtcgagttgtgcaaaaatcgttaa

SEQ ID NO: 8 - protein, 489 aa, Stevia rebaudiana
MARVDRATNLHFVLFPLLTPGHMIPMVDIARLLAERGSTVTIITTPLNANRFKPVIARAIKDRLKIQVLELKLPSTEGLPEGCENFDMIESAQFFHKMFEATYKLAEPAVNAVQRLTPPPSCIIADNLLPWTNDLAQKFKIPRIVFHGPGCFTILCIHIAMNSNVLYDIGSDSERILLPGLPDRIELTKGQALSWGRKDTKEAASFWNRVQRDEDFANGIVVNSFHALEPYYVEELAKVKGKKVWCIGPVSLCNKSFEDIAERGNKGAIDEHECLKWLDSMESRSVIFVCLGSLVRVGTEQNIDLGLGLEASKKPFLWCLRHTTEEFERWLSEQGYEERVKDRGLIIRGWAPQVFILSHRAIGGFLTHCGWNSTLEGITAGVPMVTWPQFTDQFINERFIVDVLKIGVKGGMEVPVVVGDQDKFGVLVNKEEITRSIEDLMDEGEEGETRRRRSRELRDMAKSAMEDGGSSHRDMTSMIQDIVELCKNR

SEQ ID NO: 9 - DNA, 30 bp, Saccharomyces cerevisiae
gtttcgaataaacacacataaacaaacaaa

SEQ ID NO: 10 - DNA, 30 bp, Saccharomyces cerevisiae
acaaacacaaatacacacactaaattaata

SEQ ID NO: 11 - DNA, 30 bp, Saccharomyces cerevisiae
tttcaagctataccaagcatacaatcaact

SEQ ID NO: 12 - DNA, 30 bp, Saccharomyces cerevisiae
ccaagcatacaatcaactatctcatataca

SEQ ID NO: 13 - DNA, 30 bp, Saccharomyces cerevisiae
ttatctactttttacaacaaatataaaaca

SEQ ID NO: 14 - DNA, 30 bp, Saccharomyces cerevisiae
gatacaggatacagcggaaacaacttttaa

SEQ ID NO: 15 - DNA, 30 bp, Saccharomyces cerevisiae
accatcaaaggaagctttaatcttctcata

SEQ ID NO: 16 - DNA, 1827 bp, Microcystis aeruginosa
atgtcaattaaagacacagttcaaaatacagttaatgtttctaacttttctagatcacaactaggtcaaccagatgaaaacaatttgtaccaagctgtcgcgactattacagaaggtagatggcctgaaaacttatccggttatgtgttcatcgtctgcccttttcatcgtaaaaatgataggcacttattttcaggagaaggtgttattatcaaatgggatttgcagggtaaaaacaatcaagtcaatgtgtattccaagaagttgaaaacttgggactccttctggagaaaagttttgccaatctttaacatctctcaagctacatttcctgctgtcgtgagtatattaggatgctctgaaattgctaacacagctatgataaaacttgaaaaagtagcagaggaccaacagttggaggaaacaagattgatcttgacagccgatgctggccgttactgggaagtagatccagtatctttggatactattacaccaatcgggtactttgatcaacatattgtgagcgtcccactttcattctttccagtcctggaaaataccgcccatccattttacgataaaaagacgaaggagttcatcacctgtgaattgaagctaaaactggtttctggtggtatgctcaaagatttggacaaatctgcttacattgtgttgtgggatcaacaaaagcaacttaagccttggaaactgcaaggtgccatcctggatggtagcccacactctgttatcgttaccgaagattacattatgataccagacatgccattccagatgggagttgcaaaactcctaggtatcaggataaagcctgaggaaacttaccctaagacacaaatctacctagttaaaagacaagacttaaaggaagaggaaactactgtgccatctcaactcattacattcaatggagactcttaccattttctctgtaattatcattctacaaatggtcaaatacaattagtagctattcaaaacgcaactattagtttgacagaagcgatcgaaaaggacgatatacaacattttactggacaaggctatcctcctgaataccacggcattccttggatgttttcctttgatccaggagtattgagaaaggtagtaattgaagatgcaagagttatgtctgaacaggctttcatacatccaggttggttctctacaactctctacaccgccgaccctagagaatctgagcaaggctactcagcgatctaccaggtgtatgctggatatgtcagagaactgatctgtagaaggcagtacatggattttagagatcaatcaaatagaatccttagagatgctgagttaccatgccacgatctaccatctgttctagcaaaagtctccttcgataaagactggaaccaattgacagaacaaatatcccaagagaaaaaggcctctaatactcatgtcagtcatcttggtagaggcctgttagatttttacgtttgtcctgatgggtatattctagactcaatccaattcatcccacaggagcagggctacttgttcaccactgtccttacacctactagagtgttagaagcatggttgttcaacccagataacttgaaggatggcccaattgctaaactaagtttaccagaggacgtacattttgggtttacgctgcactcagaatactttgaacaagtacttccttcaccaagaccaagtgtgtcacaagttaatcgagttttaagcgcacttagatcattagttttagtacctgttgaatttttcctaggtcgtccagcagccatctacaatagacaagttaaaaagtaa

SEQ ID NO: 17 - protein, 608 aa, Microcystis aeruginosa
MSIKDTVQNTVNVSNFSRSQLGQPDENNLYQAVATITEGRWPENLSGYVFIVCPFHRKNDRHLFSGEGVIIKWDLQGKNNQVNVYSKKLKTWDSFWRKVLPIFNISQATFPAVVSILGCSEIANTAMIKLEKVAEDQQLEETRLILTADAGRYWEVDPVSLDTITPIGYFDQHIVSVPLSFFPVLENTAHPFYDKKTKEFITCELKLKLVSGGMLKDLDKSAYIVLWDQQKQLKPWKLQGAILDGSPHSVIVTEDYIMIPDMPFQMGVAKLLGIRIKPEETYPKTQIYLVKRQDLKEEETTVPSQLITFNGDSYHFLCNYHSTNGQIQLVAIQNATISLTEAIEKDDIQHFTGQGYPPEYHGIPWMFSFDPGVLRKVVIEDARVMSEQAFIHPGWFSTTLYTADPRESEQGYSAIYQVYAGYVRELICRRQYMDFRDQSNRILRDAELPCHDLPSVLAKVSFDKDWNQLTEQISQEKKASNTHVSHLGRGLLDFYVCPDGYILDSIQFIPQEQGYLFTTVLTPTRVLEAWLFNPDNLKDGPIAKLSLPEDVHFGFTLHSEYFEQVLPSPRPSVSQVNRVLSALRSLVLVPVEFFLGRPAAIYNRQVKK

SEQ ID NO: 18 - DNA, 1833 bp, Microcystis aeruginosa
atgaacatgtcaaataaggacacagtgcaaaatactgttaacgtgtccaattttagtagaagccagctcggtagaccagatgaaaacaatctgtacaaagccgtggcaactatagccgaaggccattggccagaaaacttaagtggctatgttttcatagtctgcccttttcacagaaagaatgacagacatttgttttctggtgaaggagttatcattaagtgggatctgcaagggaaaaacaatcaagtgaatgtgtactccaaaaagctgaaaacatgggattcattttggagaaaggtgctacctatctttaacatttcacaagcaacatttcctgctgttgtctcaatcttaggttgttcagaaattgctaatactgctatggtcaaattggagaaagtcagtgaagataaacagttggaagagacaagactaattctcactgctgatgctggcagatactgggaagtagacccagtcagcctagacaccattactccaataggttactttgaccaacatattgtttccgttcctctctctattttccctgtacttgaaaatacagctcatcctttttacgacaaaaagacgcaggagttcataacatgcgaattgaaattgaagttgatctctggagggatgttaaaagatttggacaaatctgtctacatcgtattgtgggatcaacaaaagcaattaaagccttggaagctccaaggtgcaatcttggatggttctccacattcagttattgtgactgaagattacatcatgatcccagatatgccatttcaaatgggcgtcgccaaattacttgggatcagaattaaaccagaagagacttacccaaaaacccaaatatacttagtgaaaagacaagatttgaaagaggaagagactacggttccatccaggctcattacattcaatggtgactcttatcacttcctatgcaattaccactcaacaaatggtcagatccaacttgtagccatccaaaacgctacaatttcacttactgaagcaattgaaaaagacgatatccaacatttcacgggtcagggttatccacctgaataccacggtattccttggatgttctcttttgatccaggcgttttgagaaaagttgtcattgaagatgctagagttatgtctgaacaggcttttatccatccaggttggttcagtacaaccttatacacagcagatccacgtgagttggaacaaggatactctgcgatatatcaagtatatgcgggctacgttagagaactaatctgtagacgacaatatatggattgtagggatcaatctaacagaatcttacgtgatgctgaacttccttgtcatgacttgccatctgtgttggcaaaggttccattcgataaggactggaatcaattaacagaacaaatttctcaagagaaaaaggcatcagacacacatgtctcacatctgggccgtggattattggacttttacgtgtgtccagatggatatatcttagattctatacaattcataccacaagagcagggatatctgttaacaactgttttaacacctactagagtactagaagcctggttgtttaacccagataatttgaaagatggacctatcgctaagttgagcctaccagaggatgttcactttggttttaccctgcactcagagtactttgaacaggtacttccttctccaagaccatctgtttcccaagtcaatagagttttgtcagccttaaggtcccttgtattagttcctgtagaatttttcctagggaaaccagcagccatctacaacagacaagttaaaaagtaa

SEQ ID NO: 19 - protein, 610 aa, Microcystis aeruginosa
MNMSNKDTVQNTVNVSNFSRSQLGRPDENNLYKAVATIAEGHWPENLSGYVFIVCPFHRKNDRHLFSGEGVIIKWDLQGKNNQVNVYSKKLKTWDSFWRKVLPIFNISQATFPAVVSILGCSEIANTAMVKLEKVSEDKQLEETRLILTADAGRYWEVDPVSLDTITPIGYFDQHIVSVPLSIFPVLENTAHPFYDKKTQEFITCELKLKLISGGMLKDLDKSVYIVLWDQQKQLKPWKLQGAILDGSPHSVIVTEDYIMIPDMPFQMGVAKLLGIRIKPEETYPKTQIYLVKRQDLKEEETTVPSRLITFNGDSYHFLCNYHSTNGQIQLVAIQNATISLTEAIEKDDIQHFTGQGYPPEYHGIPWMFSFDPGVLRKVVIEDARVMSEQAFIHPGWFSTTLYTADPRELEQGYSAIYQVYAGYVRELICRRQYMDCRDQSNRILRDAELPCHDLPSVLAKVPFDKDWNQLTEQISQEKKASDTHVSHLGRGLLDFYVCPDGYILDSIQFIPQEQGYLLTTVLTPTRVLEAWLFNPDNLKDGPIAKLSLPEDVHFGFTLHSEYFEQVLPSPRPSVSQVNRVLSALRSLVLVPVEFFLGKPAAIYNRQVKK

SEQ ID NO: 20 - DNA, 1683 bp, Crocus sativus
atggcaaacaaagaggaagcagaaaagagaaagaagaaaccaaagcctttgaaagtactaattacaaaagtagatccaaaaccacgtaagggaatggcatctgtagctgttgatttgctagagaaagcctttgtttacttactgtacggtaattctgcggcagacagatcctctggtagacgtagacgtaaagagcactattacttatctggcaactatgctcctgtcggtcatgaaactccaccttctgaccatcttccagtgcacgggagcctgcctgaatgcttgaatggagttttcctaagagtgggtccaaatcctaagtttgctccagtcgcagggtataactgggtcgatggcgacggtatgattcatggtttgagaatcaaagatggtaaggccacttacttatccagatacatcaaaacttcaagattcaaacaagaggaatactttggtagggccaagtttatgaaaataggcgatcttagaggattactaggatttttcacaatacttatcttagttttgaggacaactttgaaggttatcgacatctcttacggtagaggcacgggtaacaccgctttagtttatcataatgggctacttttagccctctctgaggaagataaaccatacgtcgttaaagtgttggaagatggagacttacaaacgttaggtattttggactacgataaaaagttatctcatccattcactgctcatccaaaaatcgacccattaacagatgaaatgttcacattcggatactcactgtctcctccatatttgacttacagggtaatttcaaaagatggtgtgatgcaagatccagtccaaatctcaattacatctcctactataatgcatgactttgctatcaccgaaaattacgctatctttatggatcttccattgtacttccaaccagaggaaatggtgaaagggaaatttgtttcctcatttcaccctacaaaaagagctagaatcggtgttctccctagatacgcagaagatgaacatccaatcagatggtttgacctgccaagttgttttatgacccacaacgccaacgcatgggaggaaaatgatgaagtcgttttgtttacctgtcgactcgaatccccagacctggatatgttgtcaggtccagcagaagaggaaatagggaatagtaagtctgaactgtatgagatgagattcaatctcaaaacaggtataacatcccagaaacaactaagtgtaccttcagtggattttcctagaattaaccagtcatacactggtagaaagcaacaatacgtttactgtactctgggaaataccaagattaagggcattgtgaagtttgatcttcagatcgaaccagaagcgggcaaaacaatgcttgaagtaggtggcaatgtacaaggtatttttgaactaggccctcgaagatatggctctgaagctatatttgtcccatgccaacctggtatcaagagtgacgaagatgatggatatttgatctttttcgttcacgatgaaaacaatggcaagagtgaggtcaatgttattgatgctaaaacaatgtcagccgaaccagttgcagtagttcaactaccaagcagagttccttacggtttccatgctttgttccttaatgaagaggagttgcagaaacatcaagcggaaacataa

SEQ ID NO: 21 - protein, 560 aa, Crocus sativus
MANKEEAEKRKKKPKPLKVLITKVDPKPRKGMASVAVDLLEKAFVYLLYGNSAADRSSGRRRRKEHYYLSGNYAPVGHETPPSDHLPVHGSLPECLNGVFLRVGPNPKFAPVAGYNWVDGDGMIHGLRIKDGKATYLSRYIKTSRFKQEEYFGRAKFMKIGDLRGLLGFFTILILVLRTTLKVIDISYGRGTGNTALVYHNGLLLALSEEDKPYVVKVLEDGDLQTLGILDYDKKLSHPFTAHPKIDPLTDEMFTFGYSLSPPYLTYRVISKDGVMQDPVQISITSPTIMHDFAITENYAIFMDLPLYFQPEEMVKGKFVSSFHPTKRARIGVLPRYAEDEHPIRWFDLPSCFMTHNANAWEENDEVVLFTCRLESPDLDMLSGPAEEEIGNSKSELYEMRFNLKTGITSQKQLSVPSVDFPRINQSYTGRKQQYVYCTLGNTKIKGIVKFDLQIEPEAGKTMLEVGGNVQGIFELGPRRYGSEAIFVPCQPGIKSDEDDGYLIFFVHDENNGKSEVNVIDAKTMSAEPVAVVQLPSRVPYGFHALFLNEEELQKHQAET

SEQ ID NO: 22 - DNA, 3222 bp, Artificial Sequence
  Synthetic oligonucleotide encoding CYP102D1
atgactacacaacctgaaacagacctcagaccaatacgttcaccaaggggtgtcccactgttcggccacactccacaaatcccttccacaaacccagtcgaatactttggcaaactatctaaacaatttccagaaggattgtatgggatggaaattgctggtattgaacaagttttcgtatgggaccctgatttggtagctgaggtgtgcgatgaaacacgattctttaaacaaattgataaaactccacttgcacatgtcagagattacgccggcgctggtttagttacagctcatcaacacgaagaggaatggggcatggcgcatcgagtgcttttaccagtatttagccagagagctatgaaaggatactttggtcaaatgctggaaatcgcccaaaacctagttggaaaatgggaacgcaaagagggacaacctgtgaatatcacagatgattatacacgattgacactagacaccatcgccctgtcaggctttgggtacagattcgactcttttgctaaagaggatttacatccattcctaaacgccctgctccaagcattggttgaatcattgagaagatcccaggaattaccagttatgactaagatgagaaaggccgacgacaaaaagtacagagagaatatcagactaatgagagacttggttgaaaatgtgattaaggaacgtcgtgaaggcaagggcacaggtgaagatgatctattgggtcttattcttgaagctactgatcctgaaactggaaagggtttggacgacgacaatgtaagagatcaggttgttacgttcttgattgctggccacgaaacaacatctggtttattgagttttgcaacttactctctgatgagaaatccacatatccttgcccaagcgtacgcagaggtggatcgtttgcttcctggggacaccgttccagattacgataccattatgcaaatggacgtcattcctagaatcttggaggaaacgttaagattgtgggctccaatcccaatgatagggaaatctccattagaagataccgttatcggtggttgctatggtttgaaaaagggtgcaagggttaacattttagaaggacctttacatacacatcccaaggcgtgggaaagaccagaagagttcgacatcaatagatggcttccagagaatagagtgaatcatcatccacacgcttacaaaccatttggtaacggagtaagagcatgtataggtagacaatttgcattgacagaggcaagactcgctttagccttggttttacaaaaatttaagttcgccgatactgacgactataagatggatgttaaagaggccttgaccagaaagcctggtggttttgagctaaatgttagagcaagacaggaacatgaaaggaccgtctttggcgctgccgatctgcagacagatgatactcaagcccaagcagcagtttctggagtcggggttaacctgactgtagcttacggaagttcattgggttcttgcgaagatttggctcgaacaatagctgatagaggcgaaagatccggtttcgggacaacattagttggattagatgaattgggtgataatcttccaactgaaggcttgttagtggttgtcgcatcatcatacaacgggaaagcaccagataacgcacagagatttgacgatttactggctgctggattaccagaaggctctttatcaaatgtgcgtttcgccctgttaggagctggtaacacacaatgggtcgctacgtaccaaggattcccgaagagaatcgaagccgggctgttagcggctggtgcaacaagagtaattgagaggggtatcgctgatgcagccggagatttcgatggtatggccactagatggatggatacactgtggacaactcttgctgaagagtacgctgcggacacttcagaaacgaccggaccaagatttgaggtacaactattaaccgaagcagaagtgaggccagccatcgtatcagaacaagcatatccattgactgtcgttgctaatgaggaacttgtctccgatgcaactggcctctgggatttttcgatagaaccaccacgtccagcggctaagagtatcacaatagagttgccagacggtgtgacctacgatactggaaaccatttagctgtttttgccaaaaatgagcctgttctcgtaaatagggcactagccagattgggtgttgatcgtgaccaagttctgagattggatcagcccggagggggcagaacccatttgccagtaggtactcctgtcacaacaggcttactttttacagaatttgtagaattacaggatgttgctacaagatcccaaattcaggaacttgctgaacacactcagtgtccttggacaagaccacaacttcaagcgtacactgccgatacagctgaggctgaagagagataccaaaaagagattttggggaaaagagtatctgtcctaaacctattggaaagattcccagcagttgaactaccacttgcagtattccttgagatgatgggcccaattagacctagattttactccatatcatctagtcctttggccaatccaagacacgtgagattgacagtcggtctactggaaggcccagccctatctggagatggtcgttacaggggtacttgtagttcctatattgccggcctcgaaagcggtgacgtgttttatggttacgtcagagtaccttctcctacttttgcacctcctgcagatcctgctacgcctttgttgctcatcggacctggcacaggtatcgctccattgagaggtttccttgaggaaagagcacatcaacatgctcacggtactcaagtgggtctatctcaagtgttcgttggttgtagacatccagaacatgactacttttacagacaggaaatgcaagattgggaacaagcaggaattgcgcaagttcataccgctttttcagctgtcactgggcaccctgcaagattcgtccaagatgcgatagtaggcgccgctgacactgtttggcaagctatccaagacggtgcatacgtgtacgtctgtggcgatggaaggagaatggcccctgcagttagagaagctctcgctgctatctatagaaagcacacaggttctgatgatgaagctgcccaacaatggttagcacagttagaagctgacgaaagatatcaacaagatgtttttgcataa

SEQ ID NO: 23 - protein, 1073 aa, Streptomyces avermitilis
MTTQPETDLRPIRSPRGVPLFGHTPQIPSTNPVEYFGKLSKQFPEGLYGMEIAGIEQVFVWDPDLVAEVCDETRFFKQIDKTPLAHVRDYAGAGLVTAHQHEEEWGMAHRVLLPVFSQRAMKGYFGQMLEIAQNLVGKWERKEGQPVNITDDYTRLTLDTIALSGFGYRFDSFAKEDLHPFLNALLQALVESLRRSQELPVMTKMRKADDKKYRENIRLMRDLVENVIKERREGKGTGEDDLLGLILEATDPETGKGLDDDNVRDQVVTFLIAGHETTSGLLSFATYSLMRNPHILAQAYAEVDRLLPGDTVPDYDTIMQMDVIPRILEETLRLWAPIPMIGKSPLEDTVIGGCYGLKKGARVNILEGPLHTHPKAWERPEEFDINRWLPENRVNHHPHAYKPFGNGVRACIGRQFALTEARLALALVLQKFKFADTDDYKMDVKEALTRKPGGFELNVRARQEHERTVFGAADLQTDDTQAQAAVSGVGVNLTVAYGSSLGSCEDLARTIADRGERSGFGTTLVGLDELGDNLPTEGLLVVVASSYNGKAPDNAQRFDDLLAAGLPEGSLSNVRFALLGAGNTQWVATYQGFPKRIEAGLLAAGATRVIERGIADAAGDFDGMATRWMDTLWTTLAEEYAADTSETTGPRFEVQLLTEAEVRPAIVSEQAYPLTVVANEELVSDATGLWDFSIEPPRPAAKSITIELPDGVTYDTGNHLAVFAKNEPVLVNRALARLGVDRDQVLRLDQPGGGRTHLPVGTPVTTGLLFTEFVELQDVATRSQIQELAEHTQCPWTRPQLQAYTADTAEAEERYQKEILGKRVSVLNLLERFPAVELPLAVFLEMMGPIRPRFYSISSSPLANPRHVRLTVGLLEGPALSGDGRYRGTCSSYIAGLESGDVFYGYVRVPSPTFAPPADPATPLLLIGPGTGIAPLRGFLEERAHQHAHGTQVGLSQVFVGCRHPEHDYFYRQEMQDWEQAGIAQVHTAFSAVTGHPARFVQDAIVGAADTVWQAIQDGAYVYVCGDGRRMAPAVREALAAIYRKHTGSDDEAAQQWLAQLEADERYQQDVFA